(19) United States
(12) Patent Application Publication
(10) Pub. No.: US 2015/0159127 A1
(43) Pub. Date: Jun. 11, 2015

(54) MOBILE VESSEL OR CARRIER FOR THE MANUFACTURING PRODUCTION, PURIFICATION AND STERILE FILL AND FINISH OF BIOLOGICS

(71) Applicant: BioCapacity On Demand, LLC, Three Forks, MT (US)

(72) Inventors: Michael Nicholas Guerini, Gilroy, CA (US); Robert O. Keene, Three Forks, MT (US)

(21) Appl. No.: 14/621,556

(22) Filed: Feb. 13, 2015

Related U.S. Application Data
(63) Continuation-in-part of application No. 14/018,979, filed on Sep. 5, 2013.
(60) Provisional application No. 61/940,073, filed on Feb. 14, 2014.
(60) Provisional application No. 61/822,100, filed on May 10, 2013.
(60) Provisional application No. 61/837,080, filed on Jun. 19, 2013.

(51) Int. Cl. C12M 1/00; C12M 1/12

(52) U.S. Cl. CPC C12M 23/52; C12M 47/10; C12M 37/02; C12M 37/00

(57) ABSTRACT

A mobile biologics production system can include a mobile carrier configured to be trailered to a biologics production location. The mobile carrier defines an interior volume configured to provide an aseptic environment for manufacturing, production, and purification of biologics. A production sub-system disposed in the interior volume includes a single-use bioreactor configured to grow biologics which can include microbes, mammalian cell lines, insect cell lines, and plant cell lines. The bioreactor is controlled by a controller, and can have a working capacity of less than about 100 L. A purification sub-system disposed in the internal volume is fluidically coupled to the production sub-system and configured to produce a finished biologic product. A sterile fill and finish sub-system is also disposed in the interior volume defined by the mobile carrier and can be configured to produce a final Product ready for use by the end user.

This application claims priority to and the benefit of U.S. Provisional Application No. 61/940,073, filed Feb. 14, 2014, the disclosure of which is hereby incorporated by reference in its entirety.

This application is also a continuation-in-part of U.S. patent application Ser. No. 14/018,979, filed Sep. 5, 2013, which claims priority to and the benefit of U.S. Provisional Application No. 61/822,100, filed May 10, 2013, and U.S. Provisional Application No. 61/837,080, filed Jun. 19, 2013, the disclosures of which are hereby incorporated by reference in their entirety.

### BACKGROUND

Embodiments described herein relate to mobile biologics production systems that can be transported to a site where production of biologics is required and are operable to produce the biologics on-site on demand.

Biopharmaceuticals and renewable chemicals (also referred to herein as “biologics”) are a growing segment in the global biologics market. Known biologics can include, for example, veterinary proteins, human proteins, animal proteins, plant proteins, pharmaceutical proteins, renewable fuels, veterinary enzymes, human enzymes, animal enzymes, plant enzymes, pharmaceutical enzymes, microbial mass, microbial biomass, agricultural proteins, agricultural enzymes, biological control enzymes, biological control proteins, biological control microbes, virus particles, fatty acids, or bioterrorism related counter measures.

Biologics manufacturing is a technologically complicated process, which is also highly regulated, for example, by the FDA and/or the USDA. Manufacturing of biologics requires bioreactor capabilities which can produce biologics compatible with good laboratory practice (GLP) or good manufacturing practice (GMP) standards. In comparison to other types of manufacturing, biologics require far more planning, investment, documentation, skilled personnel, and regulatory approval, and therefore can be much riskier. One way biotech companies can save cost and avert risk is by contracting the manufacturing of the biologics to a biologics-focused contract manufacturing organization (CMO).

Since the late 1990's, the use of CMOs has increased steadily. However, there is no standardization among CMOs and manufacturing quality and capacity can vary greatly. While certain types of manufacturing capacity, for example, fill and finish operations are abundant, other types of capabilities, for example, up stream processes like fermentation and purification are scarce. Furthermore, supply and demand in biotech operations can be highly variable. Therefore, CMOs now represent greater than about 50% of the total microbial manufacturing capacity, and about 25% of overall mammalian cell culture capacity. As the biologics manufacturing market grows, it is expected that the demand for CMOs will continue to grow.

CMOSs can provide access to manufacturing capabilities and capacity on demand to a biologics manufacturer, without the need for investing in a brick and mortar facility. To gain widespread acceptance however, CMOs need to be able to provide biologics production systems that can easily be transported to the production site, require minimal resources and time for set up, and provide fermentation and bioreactor capabilities required by the manufacturers.

Thus, there is a need for new mobile biologics production system that can be easily transported to the site where biologics production is required, are operational for fast and cost-effective deployment, and include state of the art biologics production, purification, and sterile fill and finish capabilities.

### SUMMARY

Embodiments described herein relate to mobile biologics production systems that can be transported to a site where a production of biologics is required and are operable to produce the biologics on-site on demand. In some embodiments, a mobile biologics production system can include a mobile carrier which is configured to be trailered to a biologics production location. The mobile carrier defines an interior volume which is configured to provide an aseptic environment for manufacturing, production, purification, and sterile fill and finish of biologics. A production sub-system is also disposed in the interior volume defined by the mobile carrier. The production sub-system includes a single-use bioreactor configured to grow biologics which can include at least one of microbes, mammalian cell lines, insect cell lines, plant cell lines, and algae. The bioreactor is controlled by a controller, and can have a working capacity in the range of about 1 liter to about 100 liters. A purification sub-system is also disposed in the internal volume defined by the mobile carrier, which is fluidically coupled to the production sub-system and configured to produce a finished biologic product. A sterile fill and finish sub-system is also disposed in the internal volume defined by the mobile carrier, which is coupled to the purification sub-system and configured to produce a finished biologic product ready for dissemination to end-users. In some embodiments, the mobile carrier can be a semi-trailer.

### BRIEF DESCRIPTION OF THE DRAWINGS

FIG. 1 is a schematic block diagram of a mobile biologics production system according to an embodiment.

FIG. 2 is a side cross-section of a mobile biologics production system according to an embodiment.

FIG. 3 is a top cross-section of the mobile biologics production system shown in FIG. 2.

FIGS. 4A and 4B show side cross-section views of embodiments of mobile biologics production systems.

FIGS. 5A and 5B show top cross-section views of the mobile biologics production systems shown in FIGS. 4A and 4B, respectively.

FIGS. 6A-D shows various configurations of a mobile biologics production system that includes a plurality of mobile carriers, according to an embodiment.

FIG. 7 shows a flow diagram of a method for producing biologics on demand according to an embodiment.

FIG. 8 is a side view of an exemplary mobile biologics production system.

FIG. 9 is a top cross-section view of the mobile biologics production system of FIG. 8

FIG. 10 is a side view of an exemplary mobile biologics production system.

FIG. 11 is a top cross-section view of the mobile biologics production system of FIG. 10.

### DETAILED DESCRIPTION

Systems and methods for the production of biologics are continually advancing because of the increasing demand for capacity for manufacturing and production of biologics. This process of manufacturing biologics through the use of non-native production organisms has been termed synthetic biology. Most companies are limited by the manufacturing capacity available within their brick and mortar structure. Building of new manufacturing facilities requires significant investment and can be highly risky because of the variable demand of biologics. Therefore, the demand for mobile biologics production systems that can be transported to a site where biologics production is required is growing. Such mobile biologics production systems need to be operational on-site in a short amount of time after deployment as well as present minimum infrastructure and cost impact to the manufacturer.

Embodiments of the mobile biologics production system described herein can be transported to a site where production of biologics is required and are operable to produce the biologics on-site on demand. The mobile biologics production systems described herein provide numerous advantages, including: 1) completely pre-assembled mobile biologics production system which does not require any manufacturing or assembly on-site; 2) deployment time is short, for example, less than about 6 hours and only requires coupling of electric, and/or gas lines; 3) capability of housing bioreactors of various sizes to allow a diverse range of production volumes; 4) a plurality of the mobile biologics production system can be deployed on-site and can be coupled together such that the need for large production capacities can be met; 5) can be used for the production of biologics and/or chemicals; and 6) can produce material ready for safe and aseptic storage and use by the intended end user.

In some embodiments, a mobile biologics production system can include a mobile carrier which is configured to be trailered to a biologics production location. The mobile carrier defines an interior volume which is configured to provide an aseptic environment for manufacturing, production, and purification of biologics. A production sub-system is also disposed in the interior volume defined by the mobile carrier. The production sub-system includes a single-use bioreactor configured to grow biologics which can include microbes, mammalian cell lines, insect cell lines, plant cell lines, and algae. The bioreactors are controlled by a controller, and can have a working capacity in the range of about 1 L to about 100 L. A purification sub-system is also disposed in the internal volume defined by the mobile carrier, which is fluidically coupled to the production sub-system and configured to produce a finished biologic product. A sterile fill and finish sub-system is also disposed in the internal volume defined by the mobile carrier, which is coupled to the purification sub-system and configured to produce a finished biologic product ready for dissemination to end-users. In some embodiments, the mobile carrier can be a semi-trailer.

In some embodiments, a mobile biologics production system can include a first mobile carrier configured to be trailered to a biologics production location. The first mobile carrier defines an interior volume which is configured to provide an aseptic environment for manufacturing, production, purification, and sterile fill and finish of biologics. The mobile biologics production system also includes a second mobile carrier configured to be trailered to a biologics production location, which also defines an interior volume configured to provide an aseptic environment for manufacturing, production, purification, and sterile fill and finish of biologics. At least one of the first mobile carrier and the second mobile carrier further includes a production sub-system disposed in the interior volume defined by the first mobile carrier or the second mobile carrier. The production sub-system includes a bioreactor configured to grow at least one of microbes, mammalian cell lines, insect cell lines, plant cell lines, and algae. The bioreactors are controlled by a controller and can have a working capacity in the range of about 1 L to about 100 L. Furthermore, a purification sub-system is disposed in the interior volume defined by the first mobile carrier and/or the second mobile carrier. The purification sub-system is fluidically coupled to the production sub-system and is configured to produce a finished biologic product. A sterile fill and finish sub-system is also disposed in the internal volume defined by the mobile carrier, which is coupled to the purification sub-system and configured to produce a finished biologic product ready for dissemination to end-users.

In some embodiments, a method for producing biologics on demand can include transporting a mobile carrier to a location where production of biologics is required. The mobile carrier can include a single-use bioreactor, a controller operable to control the bioreactor, a purification sub-system, and a sub-system for the sterile fill and finish packaging of the biologic for immediate use or long-term storage. The method further includes coupling electric lines to the mobile carrier and producing biologic materials in the bioreactor. The biologic materials are transferred to a purification sub-system where they are purified. The purified biologics are then removed from the mobile carrier.

As used herein, the term “about” generally means plus or minus 10% of the value stated, e.g., about 250 would include 225 to 275, about 1,000 would include 900 to 1,100.

As used herein, the term “mobile” means a system, apparatus or device that can be transported from one location to another, but can still be operational and on demand without requiring extensive modifications or additions to the system, apparatus or device.

As used herein, the term “biologics” refers to any chemical or biochemical compound produced by a living organism which can include a prokaryotic cell line, a eukaryotic cell line, a mammalian cell line, a microbial cell line, an insect cell line, a plant cell line, a mixed cell line, a naturally occurring cell line, or a synthetically engineered cell line.

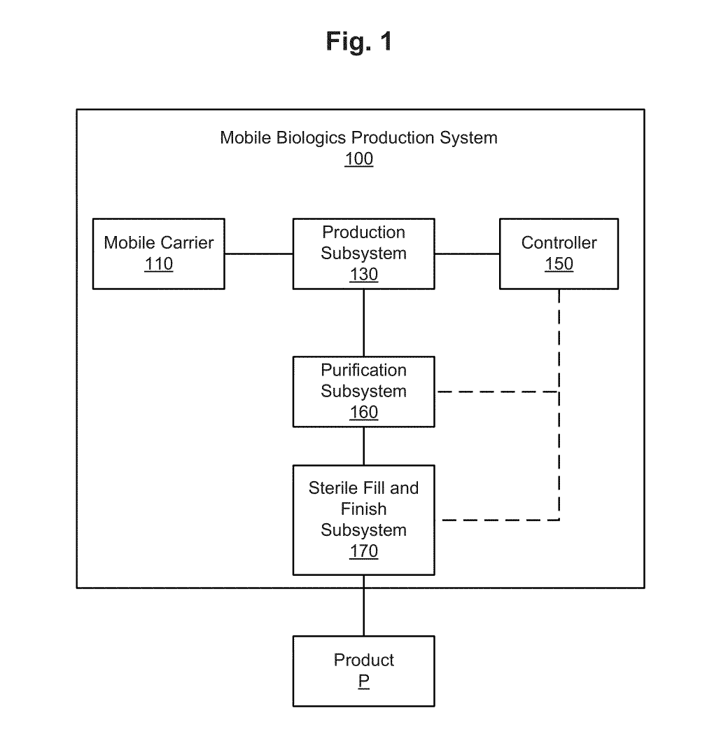

FIG. 1 shows a schematic block diagram of a mobile biologics production system 100 according to an embodiment. The mobile biologics production system 100 can include a mobile carrier 110, a production sub-system 130, a controller 150, a purification sub-system 160, and a sterile fill and finish sub-system 170. The mobile biologics production subsystem 100 can be configured to produce a finished biologic product P which can include, for example, veterinary proteins, human proteins, animal proteins, plant proteins, pharmaceutical proteins, renewable fuels, veterinary enzymes, human enzymes, animal enzymes, plant enzymes, pharmaceutical enzymes, microbial mass, microbial biomass, agricultural proteins, agricultural enzymes, biological control enzymes, biological control proteins, biological control microbes, virus particles, fatty acids, or any other biologic material. In some embodiments, the mobile biologics production system 100 can be used for the production of bioterrorism related counter measures.

The mobile carrier 110 can be a transportable carrier which can, for example, be trailered to a biologics production location. In some embodiments, the mobile carrier 110 can include, for example, a semi-trailer, a large cargo trailer, a bobtail, a gooseneck trailer (which is easily maneuverable with smaller vehicular equipment), a recreational vehicle, or any other mobile carrier. In some embodiments, the mobile carrier can be a self-propelled vehicle. The mobile carrier 110 can be a completely enclosed mobile carrier 110 which remains mobile on wheels at all times. For example, the mobile carrier can include a coupler (e.g., a king pin coupler) for coupling to a truck or a trailer, and a landing gear (not shown), such that the mobile carrier can easily be coupled to the truck or the trailer, driven to a location where biologics production is required, and deployed by engaging the landing gear. The mobile biologics production system 100 can be made operational by coupling electric, water, and/or gas lines to the mobile carrier 110 such that the mobile biologics production system 100 can be operational within a short time, for example, in less than about 6 hours from delivery. The mobile carrier 110 can ride on a heavy duty axle assembly (not shown). The axle assembly can include tires, wheels, springs and brakes (e.g., sealed electric brakes) on all axles. Furthermore, the axles can be dual and can have air ride bags.

The mobile carrier 110 defines an interior volume configured to provide an aseptic environment for manufacturing, production, purification, and sterile fill and finish of biologics. In some embodiments, the production sub-system 130, the controller 150, and the purification sub-system 160 can all be disposed within the internal volume defined by the mobile carrier 110, as described herein. In some embodiments, the purifications subsystem 160 can also be disposed in the internal volume defined by the mobile carrier 110. In some embodiments, the sterile fill and finish subsystem can also be disposed within the internal volume defined by the mobile carrier 110. The sidewalls (not shown) of the mobile carrier 110 can be made from a strong material, for example, aluminum alloy, stainless steel, or any other suitable material or combination thereof and can include, for example, roll formed “C” channels. A plurality of such channels can be joined together to form the sidewalls of the mobile carrier 110. In some embodiments, a floor of the mobile carrier 110 can be made from a plurality of “I” beams coupled together. The external sidewalls and roof of the mobile carrier 110 can be coated with a weather resistant paint, for example, PPG epoxy primers, acrylic paints, polyurethane paints, any other weather resistant paint or a combination thereof. The sidewalls can also be undercoated with a sound deadening material, for example, Quaker Koat®. In some embodiments, a roof of the mobile carrier 110 can be coated with a heat-protecting roof coating. Such a coating can reflect heat and provide a more controllable interior environment. The finished sidewalls can be relatively smooth and free of sharp edges. The sidewalls of the mobile carrier 110 can be insulated, for example, on sides, ends, floor and ceiling with a suitable insulating material (e.g., foamed-in-place urethane) to provide optimum environmental protection, add support to the sidewalls, and provide noise isolation.

In some embodiments, the mobile carrier 110 can include running light, reflectors, markers and wiring in accordance with Department of Transportation (“DOT”) or any other government entity safety regulations. In some embodiments, a plurality of windows can be included in the sidewalls. In some embodiments, mobile carrier 110 can be equipped with a full width rear bumper, for example, a steel bumper which can have reflective stripes to give an indication of the width of the mobile carrier 110. In some embodiments, a plurality of containers (not shown) can be disposed in the under carriage of the mobile carrier 110. The containers can, for example be sliding containers that can be accessed from both sides of the mobile carrier 110. The plurality of containers can, for example, be used as storage containers for storing equipment and/or supplies.

In some embodiments, a floor of the mobile carrier 110 can be coated with a non-porous material, for example, an epoxy resin, vinyl, or any other suitable material that is heavy duty and non-skid and can be cleaned easily. An interior surface of the side walls of the mobile carrier 110 can also be coated with a non-porous easily washable smooth material, for example, epoxy resin, vinyl, or acrylic paint such that the interior side walls can be easily cleaned. A drain (not shown) can be included in the floor of mobile carrier 110 to allow for draining of spills, washing, sterilizing, or decontaminating solutions from the mobile carrier 110. In some embodiments, the mobile carrier 110 can include a false ceiling which can include, for example, a plurality of panels (e.g., Formica panels) to form the false ceiling. The panels can be laminated and can include fixtures and cavities for accommodating lighting fixtures, wiring, smoke detectors, gas detectors, water sprinklers, HVAC ducts and/or equipment.

The mobile carrier 110 can also include a plurality of doors (not shown), for example, an entrance doorway, an exit doorway, an emergency exit doorway, or a connecting doorway (e.g., a doorway connecting the production sub-system to the purification sub-system). The doors can be made from a strong material, for example, aluminum, stainless steel, fiber glass or any other suitable material. The doors can include lift gate type doors, swing doors, or sliding doors, and can include glass or Plexiglas panels. A suitable access mechanism, for example, a lock and key mechanism, a pass code punch pad, card swipe, transponder reader, finger print scanner, retina scanner, or any other access mechanism, can be provided to unlock the doors. Appropriate signs, for example, biohazard, authorized personnel list, biosafety level (BSL), or any other necessary information can be posted on the doorways.

In some embodiments, the mobile carrier 110 can include a lighting sub-system configured to provide adjustable light to the interior volume defined by the mobile carrier 110. A plurality of light switches, light panels, electrical outlets (e.g., 110 V/60 Hz or 220 V/50 Hz, 15 Amp, 20 Amp, etc.) can be disposed on the interior surface of the side walls of the mobile carrier 110. The lighting sub-system can include, for example, 1, 2, or 4 tube fixture fluorescent lights mounted within plastic diffuser panels, 12 V dome lights (e.g., for mounting at entrances or exits, floodlights (e.g., for lighting exterior), LED lights, and/or one or more emergency lights. The lighting sub-system can include a rheostat configured to control the light intensity, for example, to define light/dark cycles for growing the biological material or to prevent exposure of the product or cell lines to extreme light that might otherwise damage the biologic product being produced.

In some embodiments, the mobile carrier 110 also includes an HVAC sub-system configured to provide hot and/or cool air to the interior volume defined by the mobile carrier 110, and/or provide humidity control. In some embodiments, the HVAC sub-system can include filters to purify the air coming into the interior volume. For example, HEPA filters can be included to provide a controlled amount of particulate flow to the internal volume defined by the mobile carrier 110, for example, to meet an ISO class 1,000, class 100, or class 10 cleanroom requirements. Temperature and humidity controls can also be provided in the mobile carrier 110, for example, temperature/humidity control panels can be provided on the interior side walls of the mobile carrier 110, or controlled centrally through the controller 150.

In some embodiments, the mobile carrier 110 can also include a power generation system, for example, a gas generator (e.g., a 12 kV generator), a UPS, a battery pack (e.g., li-ion battery), solar panels, wind turbines, or any other suitable power generation system. Such a power source can be used to serve as the primary or a backup power source. When a gas generator is provided, the mobile carrier can include a fuel tank (gasoline, diesel, CNG, etc.), for example, a 50 gallon fuel tank, to store the fuel for the gas generator. The power generator can be used, for example, as a backup generator to produce power for emergencies or to power the HVAC sub-system when the mobile carrier 110 is on the move. In some embodiments, the mobile carrier 110 can be provided with its own water supply. For example, the mobile carrier 110 can include a first water tank to supply clean water, a second water tank to supply filtered or deionized water, and a third tank for storage of grey water for disposal. In some embodiments, the mobile carrier 110 can also include compartments for storing gas cylinders, for example, O₂, CO₂, N₂, Air, or any other gas cylinders for providing gases needed to produce the biologics.

The interior volume defined by the mobile carrier 110 can be divided into a plurality of portions, each portion housing a different sub-system. For example, the interior volume can include a first portion housing the production sub-system 130 and the controller 150, a second portion housing the purification sub-system 160, and optionally a third portion housing a preparation area. In some embodiments, the sterile fill and finish subsystem 170 can also be included in the third portion. The preparation area can include, for example, an air shower to remove particulates, tacky mats to remove dirt from boot soles, benches (e.g., foldable benches), clothes rack and/or storage racks for storing PPEs (e.g., gloves, lab coats, booties, coveralls, face masks, hoodies, safety glasses, goggles, etc.), water fountain, UV sterilizer, or any other suitable equipment to allow a user to prepare appropriately for entry into the first portion housing the production sub-system 130 and/or the second portion housing the purification sub-system 160. In some embodiments, any of the first portion, the second portion or the third portion can include pop-out spaces that can add extra space to each of the portions when the mobile carrier 110 is stationary and be retracted when the mobile carrier 110 is moving.

The production sub-system 130 can include one or more single-use bioreactors (not shown) which are configured to grow at least one of microbes, mammalian cell lines, insect cell lines, plant cell lines, and algae. Each of the single-use bioreactors can be removably coupled to the production sub-system 130 such that the after a batch of desired biologics is produced in the bioreactors, the single-use bioreactors can be removed and replaced with a second set of single-use bioreactors. This can for example, minimize contamination. In some embodiments, multi-use bioreactors can also be used. In some embodiments, the first portion can include workbenches, for example, foldable workbenches (e.g., stainless steel benches) stowed in a side wall of the first portion that can be moved out of the way to accommodate different configurations of the production sub-system 130 or for cleaning the interior volume. The first portion can also include removable storage cabinets, for example to facilitate cleaning. Each of the bioreactors can be disposed on the work benches or on the floor. In some embodiments, eye wash stations and safety showers can also be installed in the first portion. In some embodiments, a portable UV sterilizer (e.g., a total room sanitizer) can also be included in the first portion. In some embodiments, the first portion can also include a biosafety hood, for example, to enable users to prepare seed batches of the biologic cells lines or to perform any other experimentation. In some embodiments, the first portion can also include foldable countertops, undercounter workbenches, removable bench seats, and/or flip up desks, that can be made from a smooth and easily washable material, for example, stainless steel.

Each of the single-use or multi-use bioreactor can include any suitable bioreactor, for example, perfusion bioreactors wave bioreactors, cylindrical bioreactors (e.g., 3:1 aspect ratio), bag bioreactors, moving bed bioreactors, packed bad bioreactors, fibrous bioreactors, membrane bioreactors, batch bioreactors, or continuous bioreactors. The bioreactors can be made from a suitable material, for example, stainless steel, glass, or plastic. Each of the single-use or multi-use bioreactors can have a capacity of about 1 L to about 100 L. In some embodiments, the bioreactors can have a capacity of less than about 1 L, for example about 0.5 L, about 0.1 L, about 0.05 L, about 0.01 L, or even lower, inclusive of all ranges therebetween. In some embodiments, the bioreactors can have a capacity of up to about 500 L. In some embodiments, the production sub-system 130 can include a plurality of bioreactors having different production capacities, for example, about 1 L, about 10 L, about 40 L, or about 100 L. In some embodiments, an environmentally controlled shaker can be disposed next to a small sized bioreactor (e.g., about 1 L), a medium sized bioreactor (e.g., about 10-40 L), and a large size bioreactor (e.g., about 100 L). Each of the plurality of bioreactors can also include one or more sensors, for example, a temperature sensor (e.g., a thermocouple), flow rate sensor, gas sensor, or any other sensor.

The production sub-system 130 can include a pumping sub-system (not shown) for communicating nutrients, buffers, organisms, or finished products to and from the bioreactors. For example, the pumping sub-system can include peristaltic pumps configured to communicate biologic materials from a growth vessel capable of growth as small as about 50 μL to the scaled bioreactors. For example, production of biologics (e.g., mammalian or microbial cell culture) can begin in 50 μL 96 well plates in a shaker incubator which can be placed on a work bench. The biologics can then be scaled up and pooled into flasks which range from about 10 mL to about 100 mL. The pooled biologics can then be transferred to about 1 L bioreactors, scaled pooled and transferred to about 40 L capacity bioreactors, and finally to about 100 L bioreactors. Each of the bioreactors can include electric and gas lines, which can be coupled to an external electric and/or gas supply, as described herein. In some embodiments, fermenters and/or digesters can also be coupled with the bioreactors, for example, for preprocessing nutrients supplied to a bioreactor, and/or further processing of biologics produced by the bioreactor.

The controller 150 can be configured to control the operations of the production sub-system 130, for example, the bioreactors having a working capacity in the range of about 1 L to about 100 L (e.g., about 1 L, about 10 L, about 40 L, and about 100 L). The controller 150 can include a local computer, a local server (e.g., disposed in the first portion), a remote computer, a remote server, or a network. The controller 150 can be operational to control all aspects of the biologics manufacturing process, for example, control the supply of liquid materials and/or gas communicated into the bioreactors, the lighting sub-system, the pumping sub-system, and/or the HVAC sub-system. The controller 150 can also be coupled to the sensors disposed in the bioreactors as described herein, for example, to control the temperature, volume flow rate and gas flow rate into the bioreactors in real time. In some embodiments, the controller 150 can also include a display, for example, a computer monitor, a smart phone app, a tablet app, or an analog display, that can be accessed by a user to determine the state of the production sub-system 130. In some embodiments, the controller 150 can also include an input, for example, a keyboard, a key pad, a mouse, or a touch screen, to allow a user to enter control parameters for controlling the operation of the production sub-system 130. In some embodiments, the controller 150 can also be configured to control the operation of the purification sub-system 160. In some embodiments, a single controller 150 can be used to control the operation of each of the bioreactors included in the production sub-system 130. In some embodiments, a plurality of controllers 150 can be used such that each controller 150 of the plurality of controllers 150 controls the operation of only one bioreactor.

The purification sub-system 160 can be disposed in the second portion defined by the interior volume of the mobile carrier 110. The second portion can include workbenches, for example foldable workbenches, on which the components of the purification sub-subsystem 160 can be disposed. A refrigerator, for example, to store reagents, biologic materials or finished product P can also be included in the second portion. The purification sub-system 160 can be fluidically coupled to the production sub-system 130 and can be configured to produce a finished biologic product. For example, the pumping sub-system can communicate the product produced by the production sub-system 130 which also includes impurities (e.g., buffers, cell debris, nutrients, etc.) to the purification sub-system 160. In some embodiments, the purification sub-system 160 can include a process column and/or a filtration device. For example, the purification sub-system 160 can include a chromatography column (e.g., size exclusion chromatography, ion exchange chromatography, adsorption chromatography, partition chromatography, molecular exclusion chromatography, affinity chromatography, any other suitable chromatography column, or combination thereof) for purification of the product P and/or a tangential filtration flow device for buffer exchange, dewatering, and concentration of the product P. The process column and/or filtration device can include a suitable packing material, for example, agarose gel, silica gel, silica beads, polystyrene beads, zeolite, activated carbon, or any other suitable packing material, configured to separate the product P from the impurities with high purity and integrity. In some embodiments, the pumping sub-system can also be used to pump the product P through the filtration device.

The sterile fill and finish sub-system 170 can be disposed in the third portion as defined by the interior volume of the mobile carrier 110 or can be a separate subsystem which is coupled to the mobile carrier 110. The sterile fill and finish portion can include workbenches, for example foldable workbenches, on which the components of the sterile fill and finish sub-subsystem 170 can be disposed. A refrigerator, for example, to store reagents, biologic materials or finished product P can also be included in the second portion. The sterile fill and finish sub-system 170 can be fluidically coupled to the purification sub-system 160 and can be configured to produce a finished biologic product P. For example, the pumping sub-system can communicate the product produced by the production sub-system 130 which also includes impurities (e.g., buffers, cell debris, nutrients, etc.) to the purification sub-system 160 and finally to the sterile fill and finish system 170. In some embodiments, the sterile fill and finish sub-system 170 can include systems and device for filling or packaging the finished product P. Such components can include, for example, Tubular Type I glass vials, Type II glass bottles, Cyclic Olefin Copolymer (COC) vials, or final use syringes; pumps, lypophilization equipment, cap/stopper equipment, and final packaging/wrapping equipment, any other packaging equipment or combination thereof.

Each of the production sub-system 130, the purification sub-system 160, and the sterile fill and finish sub-system 170 can be configured to produce the finished biologic product P to GLP and/or GMP standards. In some embodiments requiring further refining of product P, the production sub-system 130 and the purification sub-system 160 can be coupled to a sterile fill and finish sub-system 170 to produce the finished biologic product P to GLP, GMP, 9CFR, and 21 CFR standards. In some embodiments, the mobile biologics production system 100 can include a plurality of mobile carriers 110 that can be trailered to the site where biologics production is required. Each of the plurality of mobile carriers 110 can be deployed and coupled together to produce the product P to meet a desired production capacity.

Having described above various general principles, several exemplary embodiments of these concepts are now described. These embodiments are only examples, and many other configurations of a mobile biologics production system and/or methods for producing biologics, are contemplated.

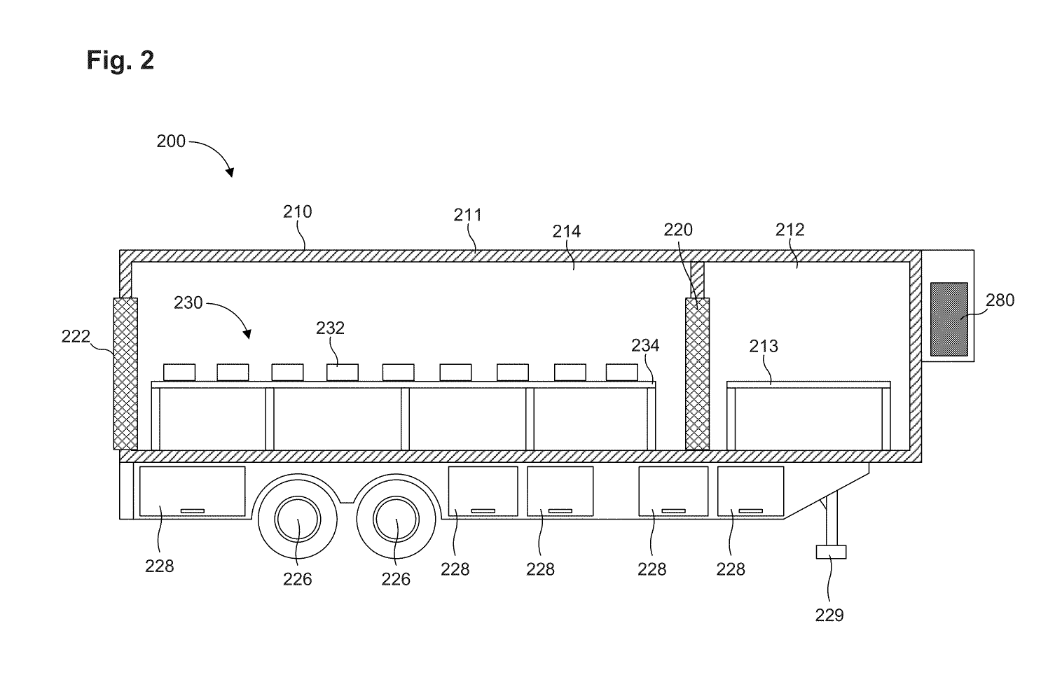

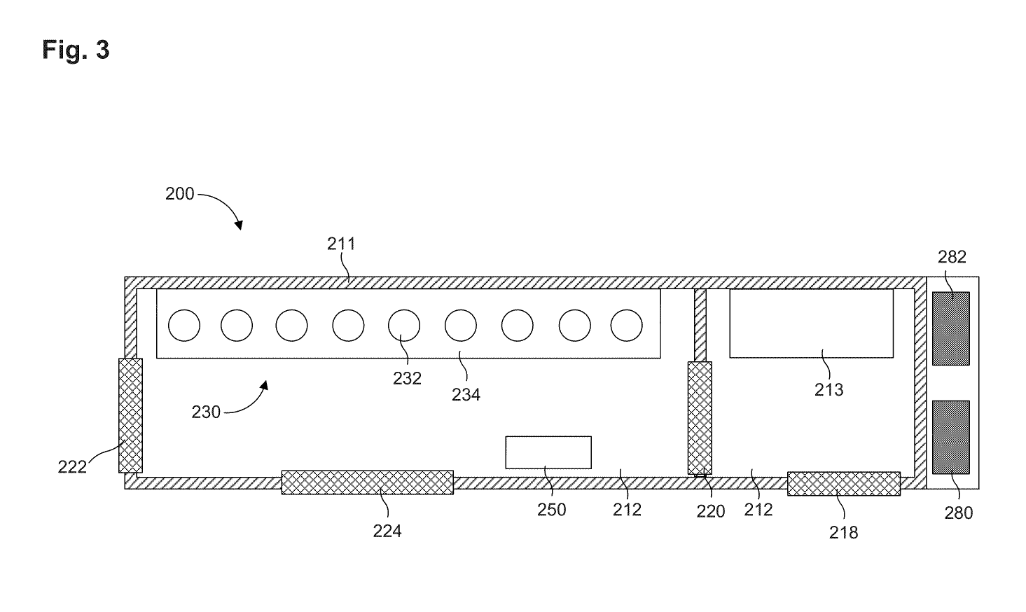

In some embodiments, a mobile biologics production system can include a preparation area and a production sub-system. Referring now to FIGS. 2 and 3, a mobile biologics production system 200 includes a mobile carrier 210, a production sub-system 230, a controller 250, and an HVAC sub-system 280. The mobile biologics production system 200 can be configured to produce a finished biologic product, for example, veterinary proteins, human proteins, animal proteins, plant proteins, pharmaceutical proteins, renewable fuels, veterinary enzymes, human enzymes, animal enzymes, plant enzymes, pharmaceutical enzymes, microbial mass, microbial biomass, agricultural proteins, agricultural enzymes, biological control enzymes, biological control proteins, biological control microbes, virus particles, fatty acids, bioterrorism related counter measures, or any other biologic product.

The mobile carrier 210 is a semi-trailer that can be trailered to a biologics production location and deployed by activating the landing gear 229. The mobile carrier 210 can be fully self contained and be made operational in a relatively short period of time, for example, less than about 6 hours from delivery, by coupling electric and/or gas lines to the mobile carrier 210. The mobile carrier 210 includes a plurality of sidewalls 211 that define an interior volume. The interior volume is divided into a first portion 212 and a second portion 214. The first portion 212 can include a preparation area, while a production sub-system 230 and a controller 250 is disposed in the second portion 214. At least the second portion 214 is configured to provide an aseptic environment for manufacturing, production and purification of biologics. In some embodiments, each of the first portion 212 and the second portion 214 can have a width of about 8 feet, a length in the range of about 2 feet to about 48 feet, and a height in the range of about 7 feet to about 9 feet. In some embodiments, the mobile carrier 210 can have a length of up to about 53 feet.

The sidewalls 211 of the mobile carrier 210 can be made from a strong material, for example, aluminum alloy, stainless steel, or any other suitable material, or combination thereof and can include, for example, roll formed “C” channels. In some embodiments, a floor of the mobile carrier 210 can be formed from a plurality of “I” beams that are coupled together. An external surface of the sidewalls 211 of the mobile carrier 210 can be coated with a weather resistant paint, for example PPG epoxy primers, acrylic paints, polyurethane paints, any other weather resistant paint or a combination thereof. The sidewalls 211 can also be undercoated with a sound deadening material, for example, Quaker Koat®. In some embodiments, a roof of the mobile carrier 210 can be coated with a heat protecting roof coating. Such a coating can reflect heat to reduce the load on the HVAC system 280.

The sidewalls 211 of the mobile carrier 210 can be made from a strong material, for example, aluminum alloy, stainless steel, or any other suitable material, or combination thereof and can include, for example, roll formed “C” channels. In some embodiments, a floor of the mobile carrier 210 can be formed from a plurality of “I” beams that are coupled together. An external surface of the sidewalls 211 of the mobile carrier 210 can be coated with a weather resistant paint, for example PPG epoxy primers, acrylic paints, polyurethane paints, any other weather resistant paint or a combination thereof. The sidewalls 211 can also be undercoated with a sound deadening material, for example, Quaker Koat®. In some embodiments, a roof of the mobile carrier 210 can be coated with a heat protecting roof coating. Such a coating can reflect heat to reduce the load on the HVAC system 270 described herein, thus providing a more controllable interior environment. Each of the sidewalls 211 can also include a plurality of windows. In some embodiments, no windows are included in the sidewalls 211. The finished sidewalls 211 can be smooth and relatively free of sharp edges. The sidewalls 211 of the mobile carrier 210 can be insulated, for example, on sides, ends, floors and ceilings with a suitable insulating material, for example, silicone, foamed-in-place urethane, fiber glass, any other insulating material or combination thereof, such that the mobile carrier 210 provides optimal environmental protection, adds support to the side-walls and provides noise isolation.

The mobile carrier 210 can include running lights, reflectors, markers, and wirings in accordance with DOT safety regulations. In some embodiments, the mobile carrier 210 can include a full width rear bumper, for example, aluminum or a steel bumper which can have reflective stripes to give an indication of the width of the mobile carrier 210 and warn other vehicles to keep a safe distance. In some embodiments, an internal surface of the sidewalls 211 can be coated with a non-porous and easily washable material, for example, epoxy resin, acrylic paint, or polyurethane paint. In some embodiments, the floor of the first portion 212 and/or the second portion 214 can be coated with a non-porous and non-skid material, for example, epoxy resin, vinyl, or any other suitable material that is heavy duty and can be cleaned easily. In some embodiments, a drain can be included in the floor of at least one of the first portion 212 or the second portion 214 to drain liquid, for example, spills, cleaning, decontamination, or sterilization liquids from the mobile carrier 210. In some embodiments, the first portion 212 and/or the second portion 214 can include a false ceiling, which can include, for example, a plurality of panels (e.g., Formica panels) to form the false ceiling. The panels can be laminated and can include fixtures and cavities for accommodating lighting fixtures, wiring, smoke detectors, gas detectors, sprinklers, HVAC ducts, and/or equipment. Fire extinguishers can also be provided in each of the first portion 212 and the second portion 214. In some embodiments, eye wash stations, safety showers, and UV sterilizers can also be included in the first portion 212 or the second portion 214.

The mobile carrier 210 includes a lighting sub-system configured to provide adjustable light to the interior volume defined by the mobile carrier 210. A plurality of light switches, panels, electrical outlets (e.g., 110 V/60 Hz, or 220 V/50 Hz, 15 Amp draw, 20 Amp draw, etc.) can be included on an interior surface of the sidewalls 211 of the mobile carrier 210. The lighting can include for example, 1, 2, or 4 tube fixture fluorescent lights mounted within plastic diffuser panels, 12 V dome lights (e.g., for mounting at entrances), floodlights (e.g., or lighting exterior), LED lights, and/or one or more emergency lights. The lighting sub-system can include a rheostat configured to control the light intensity, for example, to define light/dark cycles for growing the biological material, and/or to prevent exposure of products or cell lines to excessive light that may otherwise harm the product being produced. This can allow flexibility without needing to change the light bulbs providing rapid turnover of production.

The first portion 212 includes the preparation area which can include a plurality of benches 213, for example, foldable benches configured for sitting or performing work. The benches 213 can have adjustable heights or lengths. In some embodiments, the first portion 212 can include an air shower to remove particulates, tacky mats to remove dirt from boot soles, storage units (e.g., shelves, coat hangers, or pop out storage units) for storing personnel belongings or PPEs (e.g., gloves, lab coats, coveralls, hoodies, booties, safety glasses, goggles, facemask, etc.), water fountain, UV sterilizer, hand sanitizer, and/or any suitable equipment to allow a user to prepare appropriately for entry into the second portion 214 housing the production sub-system 230. The first portion 212 includes a first door 218 and a second door 220. The first door 218 can be a swinging door or sliding door configured to allow entry into, or exit from, the first portion 212 from the outside environment. In some embodiments, a set of steps, for example, foldable steps can be provided at the base of the first door 218 to allow a user to easily step into the first portion 212. The second door 220 can also be a swinging door or sliding door configured to allow access to the second portion 214. The second portion 214 includes a third door 222 and a fourth door 224. The third door 222 can be a swinging door or a sliding door which is configured to allow access from the second portion 214 into the outside environment. In some embodiments, a set of steps, for example, foldable steps can also be provided at the base of the third door to allow a user to easily step into the outside environment from the second portion 214. The fourth door 224 can be a lift door to also allow access into the second portion 214 form the outside environment, for example, to dispose or replace bioreactors 232 from the second portion 214. In some embodiments, a sliding ramp can be included at a base of the fourth door 224 for easy loading or unloading of materials from the second portion 214. Each of the doors can be made from a strong material, for example, aluminum, stainless steel, fiber glass, any other suitable material, or combination thereof. Each door can also include glass or Plexiglas panels. At least one of the first door 218, the second door 220, the third door 222, or the fourth door 224 can include a suitable access mechanism, for example, a lock and key mechanism, a pass code punch pad, a card swipe, a transponder reader, a fingerprint scanner, a retina scanner, or any other suitable access mechanism to limit access to at least one of the first portion 212 and/or the second portion 214 to authorized personnel.

The mobile carrier 210 rides on a heavy duty dual axle assembly 226 which includes tires, wheels, springs and brakes, for example, sealed electric brakes. The axle assembly 226 can also include air ride bags. A plurality of containers 228 are disposed in the undercarriage of the mobile carrier 210. Each of the containers 228 can be a sliding container that can be accessed from both sides of the mobile carrier 210, and can be used as storage containers for storing equipment and/or accessories. In some embodiments, each of the plurality of containers 228 can be up to about 8 feet wide and up to about 2 feet high. In some embodiments, a plurality of pop out spaces can be included in the first portion 212 and/or the second portion 214 to add extra space. The pop-out spaces can be engaged when the mobile carrier 210 is stationary, and stowed when the mobile carrier 210 is moving.

The production sub-system 230 includes a plurality of single-use bioreactors 232 which are configured to grow at least one of microbes, mammalian cell lines, insect cell lines, plant cell lines, and algae. Each of the single-use bioreactors 232 can be removably coupled to the production sub-system 230 such that after a batch of desired biologics is produced, the single-use bioreactors 232 can be replaced with a new set of single-use bioreactors 232. This can minimize contamination. In some embodiments, the bioreactors 232 can be multi-use bioreactors. The second portion 214 can include a plurality of workbenches 234 on which the single-use bioreactors 232 and/or other equipment can be disposed. The workbenches 234 can be, for example, foldable workbenches which can be stowed in a sidewall 211 of the second portion 214. The workbenches can be made from a smooth and easily washable material, for example, stainless steel. In some embodiments, each of the single-use bioreactors 232 can be disposed on the floor of the second portion 214. In some embodiments, the second portion 214 can also include benches for sitting, for example, stools or foldable benches with adjustable heights and/or lengths, stainless steel countertops, and/or flip up desks.

Each of the single-use bioreactor 232 can include any suitable bioreactor, for example, a perfusion bioreactor, a wave bioreactor, a cylindrical bioreactor (e.g., 3:1 aspect ratio), a bag bioreactor, a moving bed bioreactor, a packed bad bioreactor, a fibrous bioreactor, a membrane bioreactor, a batch bioreactor, or a continuous bioreactor. The bioreactors 232 can be made from a suitable material, for example, stainless steel, glass, or plastic. Each of the single-use bioreactor 232 can have a capacity in the range of about 1 L to about 100 L. In some embodiments, the bioreactors 232 can have a capacity of less than about 1 L, for example about 0.5 L, about 0.1 L, about 0.05 L, about 0.01 L, or even lower inclusive of all ranges therebetween. In some embodiments, the bioreactors 232 can have a capacity of up to about 500 L. In some embodiments, the production sub-system 230 can include a plurality of bioreactors 232, each having a different production capacity, for example, about 1 L, about 10 L, about 40 L, and/or about 100 L. In some embodiments, an environmentally controlled shaker can be disposed next to a small sized bioreactor (e.g., about 1 L), a medium sized bioreactor (e.g., about 10-40 L), and a large size bioreactor (e.g., about 100 L). Each of the plurality of bioreactors 232 can include one or more sensors, for example a temperature sensor (e.g., a thermocouple probe), a flow rate sensor, a gas sensor, or any other sensor.

The production sub-system 230 can also include a pumping sub-system for fluidically communicating materials, solutions, organisms, and/or finished products to and from the bioreactors 232. For example, the pumping system can include peristaltic pumps configured to communicate biologic materials from growth vessels capable of growth as small as about 50 μL to the bioreactors 232. For example, production of biologics (e.g., mammalian or microbial cell culture) can be in 50 μL 96 well plates in a shaker incubator. The biologics can be pooled into flasks which range form about 10 mL to about 100 mL. The pooled biologics can then be transferred, for example, fluidically communicated using the pumping sub-system to about 1 L bioreactors 232, scaled, pooled and transferred to about 40 L bioreactors 232, and finally to about 100 L bioreactors 232. Each of the bioreactors 232 can include electric and gas lines which can be coupled to an external electric and gas supply respectively, as described herein. In some embodiments, fermenters and/or digesters can also be coupled with the bioreactors 232, for example, for preprocessing nutrients supplied to a bioreactor 232 and/or further processing of products produced by the bioreactors 232.

The controller 250 can be configured to control the operation of the production sub-system 230, for example, the bioreactors 232 having a capacity in the range of about 1 L to about 100 L (e.g., about 1 L, about 10 L, about 40 L, and about 100 L). The controller 250 can include a local computer or a local server which can be disposed in the second portion 214, as shown in FIG. 3. In some embodiments, the controller 250 can be a remote computer and or a remote server such that the controller 250 can be disposed at a remote location to control the operations of the production sub-assembly 230 remotely, for example, from a brick and mortar facility. In some embodiments, the controller 250 can be coupled to a network, such that the controller 250 can be operated from multiple locations or can be used to control a plurality of mobile carriers. The controller 250 can be operable to control all aspects of the biologics manufacturing process, for example, control the lighting sub-system, the pumping sub-system, and/or control the supply of liquids and/or gases into the bioreactors 232. The controller 250 can also be coupled to the sensors disposed in the bioreactors 232 as described herein, for example, to control the temperature, volume flow rate and/or gas flow rate in the bioreactors 232 in real time. In some embodiments, the controller 250 can also include a display, for example, a computer monitor, a smart phone app, a tablet app, or an analog display that can be accessed by a user to determine the state of the production sub-system 230. In some embodiments, the controller 250 can also include an input, for example, a keyboard, a keypad, a mouse, or a touch screen to allow the user to enter control parameters for controlling the operation of the production sub-assembly 230. The finished product produced by the production sub-assembly 230 can be communicated to a purification sub-assembly located outside the mobile carrier 210, for example, at a brick and mortar facility. In some embodiments, the purification sub-assembly can be substantially similar to the purification sub-assembly 160 described with reference to FIG. 1, or any other purification sub-assemblies described herein. In some embodiments, a single controller 250 can be used to control the operation of each of the bioreactors 232 included in the production sub-system 230. In some embodiments, a plurality of controllers 250 can be used such that each controller 250 of the plurality of controllers 250 controls the operation of only one bioreactor 232.

The mobile carrier 210 also includes an HVAC sub-system 280 configured to provide temperature, humidity and particulate controlled air to the first portion 212 and the second portion 214. The HVAC sub-system 280 can be disposed in a front portion of the mobile carrier as shown in FIGS. 2 and 3, but in some embodiments, the HVAC sub-system 280 can be disposed in any other suitable location of the mobile carrier 210, for example the under-carriage or on the roof of the mobile carrier 210. In some embodiments, the HVAC sub-system 280 can include filters to purify the air flowing into the first portion 212 and/or the second portion 214. In some embodiments, the HVAC sub-system 280 can include HEPA filters, for example, disposed in the second portion 214 which houses the production sub-system 230. The HVAC sub-system 280 can thereby be used to provide a controlled amount of particulate flow, for example, to meet an ISO class 1,000, class 100, or class 10 cleanroom environment around the production sub-system 230. Temperature and humidity controls can be provided on the interior surface of the sidewalls 211 of the mobile carrier 210. In some embodiments, the HVAC sub-system 280 can be controlled centrally by the controller 250.

The mobile carrier 210 also includes a power generator 272, for example, a gas generator (e.g., a 12 kV generator), a UPS, a battery pack (e.g., li-ion or lead-acid), solar panels, wind turbines, or any other suitable power generation system. The power generator 272 can be disposed in a front portion of the mobile carrier 210 as shown in FIG. 3, or any other suitable portion of the mobile carrier 210, for example, in the undercarriage. In some embodiments, the power generator 272 can be used as a backup power source, for example, when external electric supply is disconnected because of an emergency situation or during transportation of the mobile carrier 210. In some embodiments, the generator 272 can also be used as the primary power supply for the mobile biologics production system 200. In some embodiments, when the power generator 272 is a gas generator, the mobile carrier 210 can also include a fuel tank, for example, a 50 gallon tank to store the fuel for the gas generator.

In some embodiments, the mobile biologics production system 200 can include its own water supply. For example, the mobile carrier 210 can include a first water tank to supply clean water, a second water tank to supply de-ionized or filtered water, and a third tank for storage of grey water for disposal. In some embodiments, the mobile carrier 210 can also include compartments for storing gas cylinders, for example, O₂, CO₂, N₂, H₂, air, and/or any other gas cylinders for providing gas needed to produce the biologics, or any other purpose, such that no external gas connections are required.

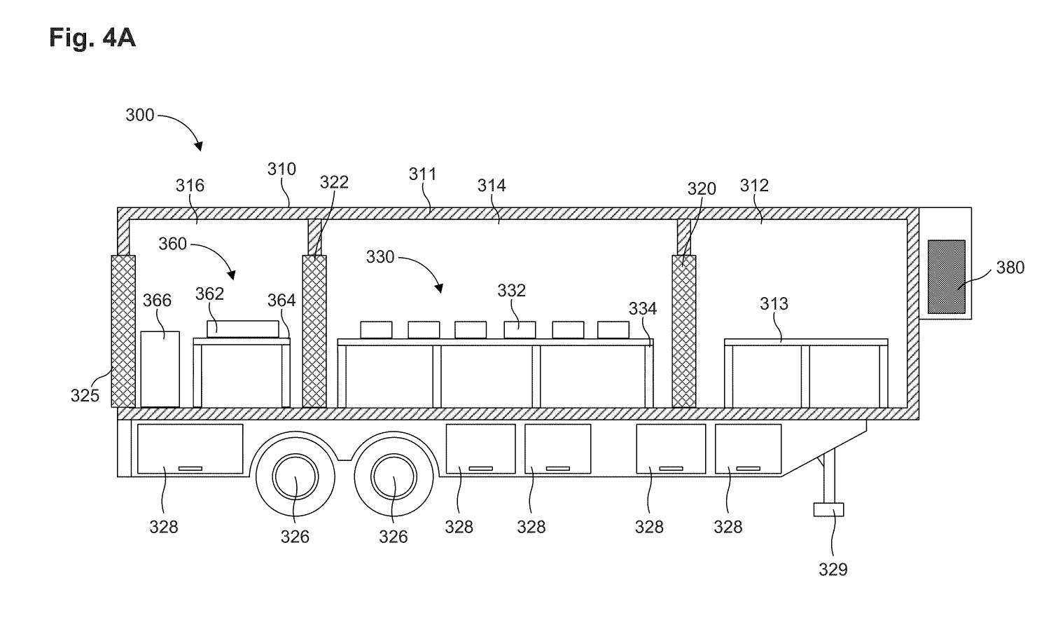

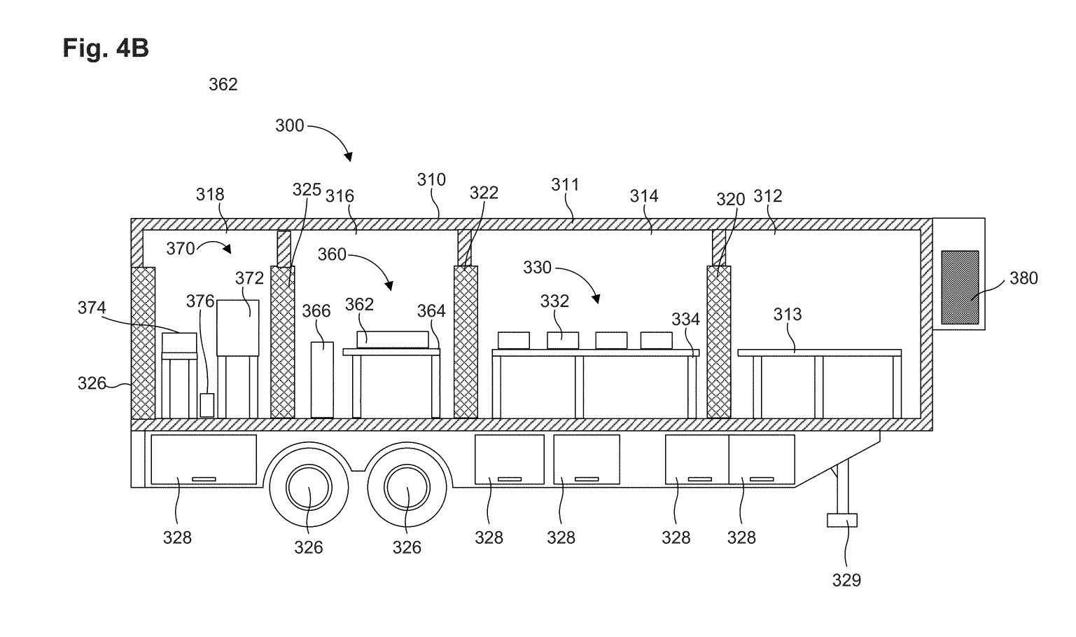

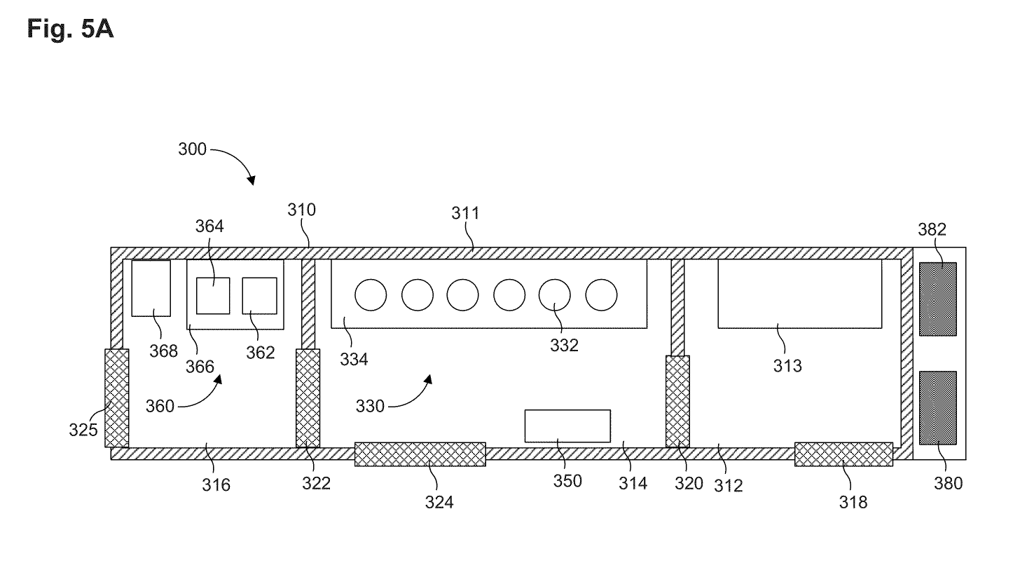

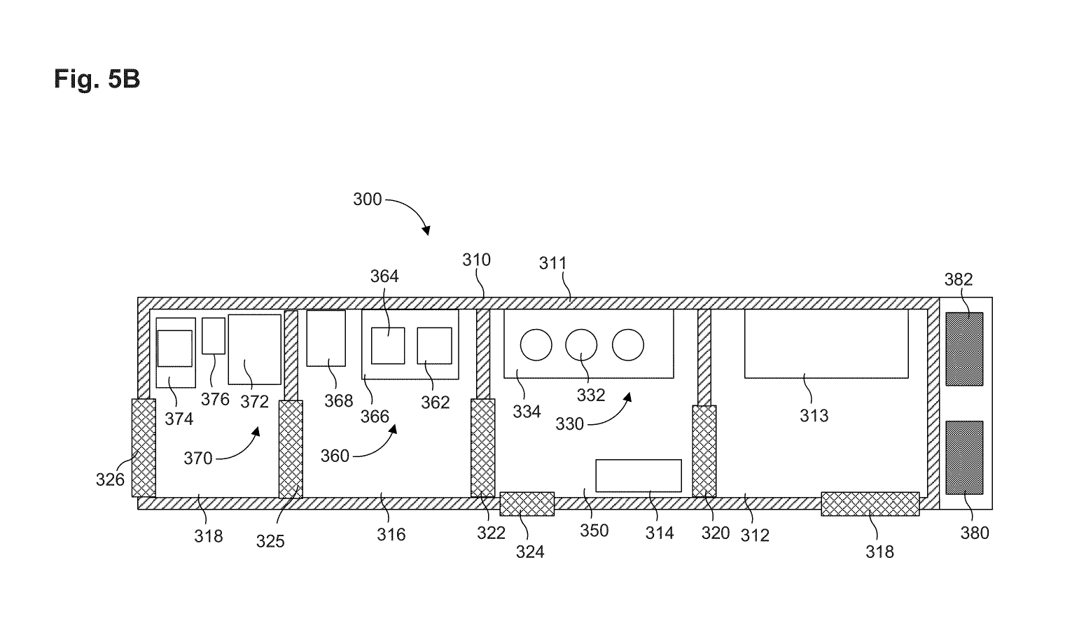

In some embodiments, a mobile biologics production system can also include a purification sub-system. Referring now to FIGS. 4A and 5A, a mobile biologics production system 300 includes a mobile carrier 310, a production sub-system 330, a controller 350, a purification sub-system 360, and an HVAC sub-system 380. In some embodiments, a sterile fill and finish subsystem 370 can also be included in the mobile production subsystem 300, as shown in FIGS. 4B and 5B. The mobile biologics production system 300 can be configured to produce a finished purified biologic product, for example, veterinary proteins, human proteins, animal proteins, plant proteins, pharmaceutical proteins, renewable fuels, veterinary enzymes, human enzymes, animal enzymes, plant enzymes, pharmaceutical enzymes, microbial mass, microbial biomass, agricultural proteins, agricultural enzymes, biological control enzymes, biological control proteins, biological control microbes, virus particles, fatty acids, bioterrorism related counter measures, or any other biologic product.

The mobile carrier 310 includes a semi-trailer that can be trailered to a biologics production location and deployed by activating the landing gear 329. The mobile carrier 310 can be fully self contained and be made operational in a relatively short period of time, for example, less than about 6 hours after deployment, by coupling electric, water, and/or gas lines to the mobile carrier 310. The mobile carrier 310 includes a plurality of sidewalls 311 that define an interior volume. The sidewalls 311 can also define a floor and a ceiling of the mobile carrier 310. The sidewalls 311 of the mobile carrier 310 can be substantially similar to the sidewalls 211 of the mobile carrier 210 and are therefore, not described herein in further detail. The sidewalls 311 of the mobile carrier 310 can be insulated, for example, on sides, ends floors, and ceilings with a suitable insulating material, for example, silicone, foamed-in-place urethane, fiber glass, any other insulating material or combination thereof, such that the insulating material provides optimal environmental protection, adds support to the sidewalls 311 and provides noise isolation. The mobile carrier 310 can include running lights, reflectors, markers, and wirings in accordance with DOT safety regulations. In some embodiments, the mobile carrier 310 can include a full width rear bumper, for example, an aluminum or a steel bumper which can have reflective stripes to give an indication of the width of the mobile carrier 310 and warn other vehicles to keep a safe distance.

The interior volume of the mobile carrier 310 is divided into a first portion 312, a second portion 314 and a third portion 316 (FIGS. 4A and 5A). In some embodiments, the interior volume of the mobile carrier 310 is divided into a first portion 312, a second portion 314, a third portion 316 and a fourth portion 318 (FIGS. 4B and 5B). The first portion 312 includes a preparation area, the second portion 314 houses the production sub-system 330 and the controller 350, the purification sub-system 360 is disposed in the third portion 316, and the sterile fill and finish sub system 370 is housed in the fourth portion 318. The first portion 312, the second portion 314, the third portion 316, and/or the fourth portion 318 can be configured to provide an aseptic environment for the manufacturing, production, and purification of biologics. Furthermore, the production sub-system 330, the purification sub-system 360, and the sterile fill and finish sub-system 370 can be configured to produce the finished biologic product up to GLP and/or GMP standards. In some embodiments, each of the first portion 312, the second portion 314, the third portion 316, and/or the fourth portion 318 can have a width of about 8 feet, a length in the range of about 2 feet to about 48 feet, and a height in the range of about 7 feet to about 9 feet. In some embodiments, the mobile carrier 310 can have a length of up to about 53 feet. In some embodiments, fire extinguishers, eye wash stations and safety showers can also be provided in at least one of the first portion 312, the second portion 314, the third portion 316, and the fourth portion 318.

The mobile carrier 310 includes a lighting sub-system configured to provide adjustable light to each of the first portion 312, the second portion 314 and the third portion 316 of the mobile carrier 310. The lighting sub-system can be substantially similar to the lighting sub-system included in the mobile carrier 210, and is therefore, not described herein in further detail. The first portion 312 includes a preparation area which can include a plurality of benches 313, for example, foldable benches configured for sitting or performing work. The first portion 312 housing the preparation area can be substantially similar to the first portion 212 included in the mobile carrier 210 and is therefore, not described herein in further detail. The first portion 312 includes a first door 318 and a second door 320. The first door 318 can be a swinging door or a sliding door configured to provide entry into or exit from the first portion 318 into the outside environment. In some embodiments, a set of steps, for example, foldable steps, can be provided at the base of the first door 318 to allow a user to easily step into the first portion 212. The second door 320 can also be a swinging door or sliding door configured to provide access to the second portion 314. The second portion 314 includes a third door 322 and a fourth door 324. The third door 322 can be a swinging door or a sliding door which is configured to allow access from the second portion 314 into the third portion 316. The fourth door 324 can be a lift door configured to allow access into the second portion 314 from the outside environment, for example, to remove or replace bioreactors 332 included in the production sub-system 330, from the second portion 314. In some embodiments, a sliding ramp can be included at a base of the fourth door 324, for example, to allow easy loading or unloading of materials from the second portion. The third portion 316 includes a fifth door 325. The fifth door 325 can be a swinging door or a sliding door and is configured to provide access to the outside environment. In some embodiments. The fourth portion 318 may also contain a door 326 that can be a swinging door or a sliding door and is configured to provide access to the outside environment. In some embodiments, a set of steps, for example, foldable steps can be provided at the base of the fifth door 325 to allow a user to easily step into the outside environment from the third portion 316. Each of the doors can be made from a strong material, for example, aluminum, stainless steel, fiber glass, any other suitable material or combination thereof. Each door can also include glass or Plexiglas panels. At least one of the first door 318, the second door 320, the third door 322, the fourth door 324, the fifth door 325, and/or the sixth door 326 can include a suitable access mechanism, for example, a lock and key mechanism, a pass code punch pad, a card swipe, a transponder reader, a finger print scanner, a retina scanner, or any other access mechanism, to limit access to the first portion 312, the second portion 314, the third portion 316, and/or the fourth portion 318.

The mobile carrier 310 rides on a heavy duty axle assembly 326 which includes tires, wheels, springs and brakes, for example, sealed electric brakes. The axle assembly 326 can also include air ride bags. A plurality of containers 328 are disposed in the undercarriage of the mobile carrier 310. Each of the containers 328 can be a sliding container that can be accessed from both sides of the mobile carrier 310, and can be used as a storage container for storing equipment and/or supplies. In some embodiments, each of the plurality of containers 328 can be up to about 8 feet wide and up to about 2 feet high. In some embodiments, a plurality of pop-out spaces can be included in the first portion 312, the second portion 314, and/or the third portion 316 to add extra space. The pop-out spaces be engaged when the mobile carrier 310 is stationary, and stowed when the mobile carrier 310 is moving.

The production sub-system 330 can include a plurality of single-use bioreactors 332 which are configured to grow at least one of microbes, mammalian cell lines, insect cell lines, plant cell lines, and algae. In some embodiments, the bioreactors 332 can be multi-use bioreactors. The second portion 314 also includes a plurality of workbenches 334, for example, foldable benches on which the single-use bioreactors 332 and/or other equipment can be disposed. In some embodiments, the second portion 314 can also include benches 334 for sitting, for example, stools or foldable benches with adjustable heights and/or lengths. The production sub-system 330 can also include a pumping sub-system for fluidically communicating materials, solutions, organisms, and/or finished product to and from the bioreactors 332. The production sub-system 330 and the pumping sub-system included therewith can be substantially similar to the production sub-system 230 and the pumping sub-system described herein with reference to the mobile biologics production system 200, and is therefore not described in further detail herein.

The controller 350 can be configured to control the operation of the production sub-system 330, for example the bioreactors 332 which can have a capacity in the range of about 1 L to about 100 L (e.g., about 1 L, about 10 L, about 40 L, or about 100 L). The controller 350 can be substantially similar to the controller 250 included in the mobile biologics production system 200 and is therefore, not described herein in further detail.

The unpurified product produced by the production sub-system 330 is communicated to the purification sub-system 360, for example, by the pumping sub-system to produce a finished biologic product. The purification can include removal of impurities, for example, particulates, cell debris, trace amount of other biologics, buffers, salts, nutrients, etc. The purification sub-system 360 can include a process column 362 and/or a filtration device 364. The process column 362 can include a chromatography column such as, for example, a size exclusion, an ion exchange, an adsorption, a partition, a molecular exclusion, an affinity, or any other suitable chromatography column, or combination thereof, which can be used to increase the purity of the product, for example, up to about 99% purity. The filtration device 364 can include, for example, a tangential flow filtration device for clarifying or concentrating biologics, dewatering, and/or buffer exchange. Each process column 362 and/or filtration device 364 can include a suitable packing or filtration material, for example, agarose gel, silica gel, silica beads, polystyrene beads, zeolite, activated carbon, membranes, or any other suitable packing material configured to the purify the product to produce a finished biologic product with high purity and integrity. In some embodiments, the pumping sub-system can also be used to pump the product through the purification sub-system 360. A plurality of workbenches 366, for example, foldable workbenches can be disposed in the third portion 316, on which the process column 362 and/or the filtration device 364 can be disposed. A refrigerator 368 can also be disposed in the third portion 316 which can be used, for example, to store reagents, biologic materials, and/or finished products.

The purified product produced by the purification sub-system 360 can be transferred to the sterile fill and finish sub-system 370. The sterile fill and finish sub-system 370 can dispose purified product into bottles, tubes, syringes, vials, jars, or any other containers for use by an end user. The sterile fill and finish sub-system 370 can include a sterile hood 372, a bottle sealing/capping device 374, and/or a lypophilizer 376. In some embodiments, one or more workbenches, for example, foldable workbenches can be disposed in the fourth portion 318, on which the sealing/capping device 374 can be disposed. A refrigerator can also be disposed in the fourth portion 318 which can be used, for example, to store packaged product ready for delivery to the end user.

The mobile carrier 310 also includes an HVAC sub-system 380 configured to heat and/or cool the air supplied into the interior volume of the mobile carrier 310, control the humidity, and/or purify the air in the interior volume. The HVAC sub-system 380 can be disposed in a front portion of the mobile carrier 330 (FIGS. 4-5). In some embodiments, the HVAC sub-system 380 can be disposed in any other suitable location in the mobile carrier 330, for example, the undercarriage or on the roof. In some embodiments, the HVAC sub-system 380 can include filters to purify the air flowing into the first portion 312, the second portion 314, and/or the third portion 316. In some embodiments, the HVAC sub-system 380 can include HEPA filters disposed in the second portion 314 and/or the third portion 316. The HVAC sub-system 380 can thereby be used to provide a controlled amount of particulate flow, for example, to meet an ISO class 1,000, class 100, or class 10 cleanroom environment around the production sub-system 330 and/or the purification sub-system 360. Temperature and humidity controls can also be provided on the interior surface of the sidewalls 311 of the mobile carrier 310. In some embodiments, the HVAC sub-system 380 can be controlled centrally by the controller 350.

The mobile carrier 310 can also include a power generator 382 for providing primary or back up power to the mobile biologics production system 300. The power generator 382 can be disposed in the front portion of the mobile carrier 310 (FIGS. 5A and 5B) or any other suitable portion of the mobile carrier 310, for example, the undercarriage. The power generator 382 can be substantially similar to the power generator 272 described with reference to the mobile biologics production system 200 and is therefore, not described herein in further detail.

In some embodiments, a mobile biologics production system can include a plurality of mobile carriers which are coupled together to provide a desired production capacity of finished biologics. In some embodiments, a mobile biologics production system can include a first mobile carrier, for example the mobile carrier 100, 200, 300 or any other mobile carrier described herein, configured to be trailered to a biologics production location. The first mobile carrier can define an interior volume which is configured to provide an aseptic environment for manufacturing, production, and purification of biologics. The mobile biologics production system also includes a second mobile carrier, for example, the mobile carrier 100, 200, 300 or any other mobile carrier described herein, configured to be trailered to the biologics production location. The second mobile carrier can also define an interior volume configured to provide an aseptic environment for manufacturing, production, and purification of biologics. The second mobile carrier is configured to be coupleable to the first mobile carrier, for example, through coupling of electrical, water, and gas lines, and/or physical coupling through a temporary walkway (e.g., an enclosed walkway). A production sub-system, for example, the production sub-system 130, 230, 330 or any other production sub-system described herein, can be disposed in at least one of the interior volumes defined by the first mobile carrier and/or the second mobile carrier. The production sub-system can include one or more bioreactors, for example, the bioreactors 232, 332, or any other bioreactors described herein, configured to grow at least one of microbes, mammalian cell lines, insect cell lines, and/or plant cell lines.

The mobile biologics production system can also include a controller, for example, the controller 150, 250, 350, or any other controller described herein, which is operable to control the bioreactors having a working capacity in the range of about 1 L to about 100 L (e.g., about 1 L, about 10 L, about 40 L, and about 100 L). In some embodiments, the first mobile carrier can have a first controller disposed in the interior volume of the first mobile carrier, and a second controller can be disposed in the internal volume defined by the second mobile carrier. The first controller and the second controller can be configured to control the operation of the bioreactors disposed in the first mobile carrier and the second mobile carrier, respectively. In some embodiments, the mobile biologics production system can include a central controller for controlling the operation of the bioreactors disposed in the first mobile carrier as well as the second mobile carrier. The central controller can be disposed in any one of the first mobile carrier or the second mobile carrier or at a remote location. In some embodiments, the controller can be a remote computer or server which is connected via a network (e.g., LAN, WAN, Wi-Fi, etc.) to local terminals located in each of the first mobile carrier and/or the second mobile carrier. This can allow for local as well as centralized control of the operation of the bioreactors in each of the first mobile carrier and/or the second mobile carrier. In some embodiments, a plurality of controllers can be used such that each controller controls the operation of only one bioreactor.

A purification sub-system, for example, the purification sub-system 160, 260, 360, or any other purification sub-system described herein, can be disposed in at least one of the interior volumes defined by the first mobile carrier and the second mobile carrier. The purification sub-system can be fluidically coupled to the production sub-system and is configured to produce a finished biologic product, for example, veterinary proteins, human proteins, animal proteins, plant proteins, pharmaceutical proteins, renewable fuels, veterinary enzymes, human enzymes, animal enzymes, plant enzymes, pharmaceutical enzymes, microbial mass, microbial biomass, agricultural proteins, agricultural enzymes, biological control enzymes, biological control proteins, biological control microbes, virus particles, fatty acids, bioterrorism counter measures, or any other finished biologic product described herein.

A sterile fill and finish sub-system, for example sub-system 170, 370, or any other sterile fill and finish sub-system described herein, can be disposed in at least one of the interior volumes defined by the first mobile carrier and the second mobile carrier. The sterile fill and finish sub-system can be used to produce finished product P that includes for example, veterinary proteins, human proteins, animal proteins, plant proteins, pharmaceutical proteins, renewable fuels, veterinary enzymes, human enzymes, animal enzymes, plant enzymes, pharmaceutical enzymes, microbial mass, microbial biomass, agricultural proteins, agricultural enzymes, biological control enzymes, biological control proteins, biological control microbes, virus particles, fatty acids, bioterrorism counter measures, or any other finished biologic product described herein. These finished products P produced in the sterile fill and finish subsystem can be directly used by end users.

In some embodiments, each of the first mobile carrier and the second mobile carrier can include a preparation area, for example, the preparation area described with respect to the mobile biologics production system 200, 300, or any other preparation area described herein, a production sub-subsystem (e.g., the production sub-system 130, 230, or 330), a purification sub-system (e.g., the purification sub-system 160, or 360), and a sterile fill and finish sub-system (e.g., the purification sub-system 170, or 370). In some embodiments, the first mobile carrier and the second mobile carrier can be dedicated to a particular sub-system. For example, the first mobile carrier can include the production sub-system and/or the preparation area, and the second mobile carrier can include the purification sub-system and/or the sterile fill and finish subsystem. In some embodiments, a third mobile carrier can be coupled to the first mobile carrier, such that the third mobile carrier includes only the preparation room.

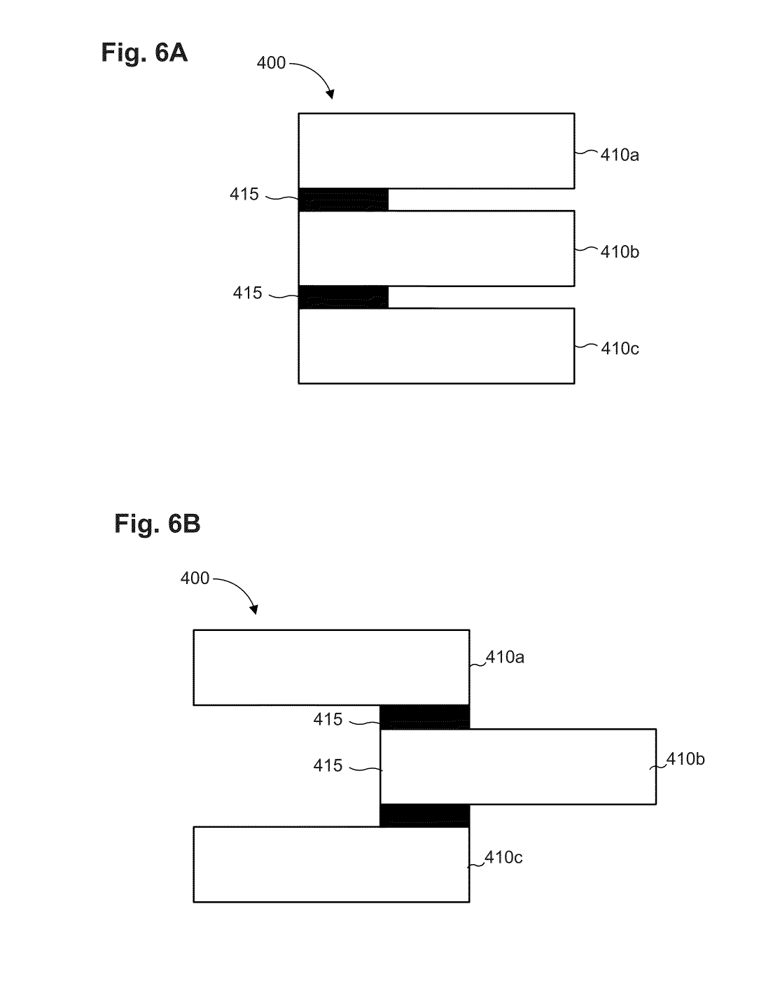

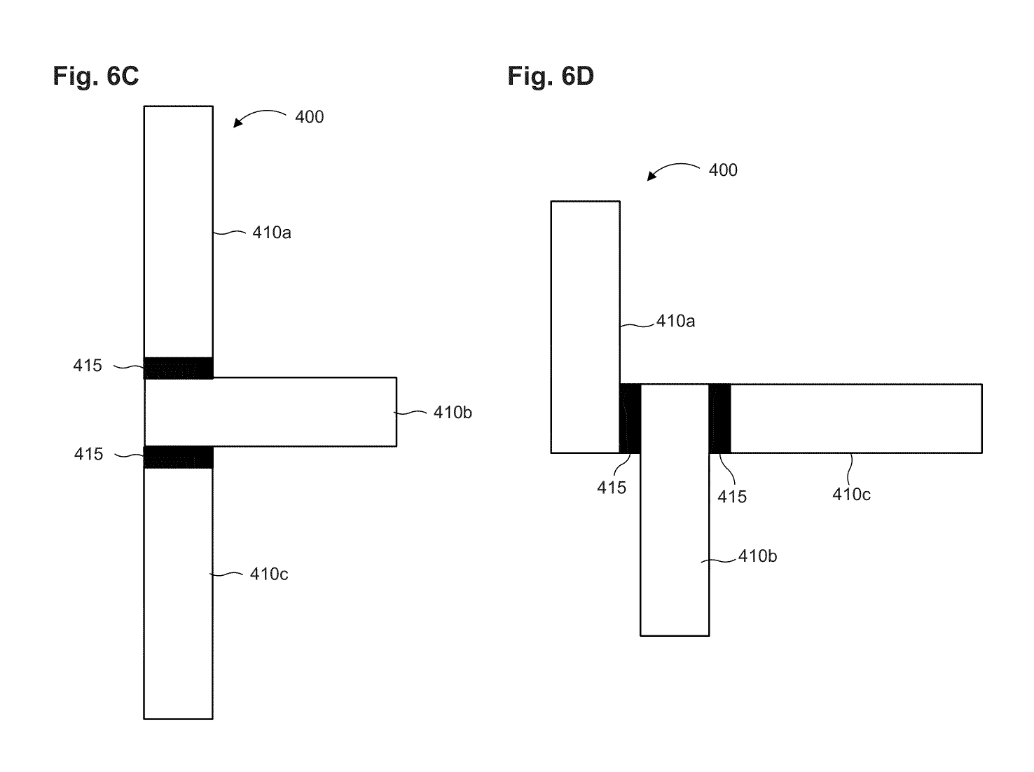

Any number of mobile carriers described herein can be coupled together to realize a mobile biologics production system of desired working capacity. The one or more mobile carriers can be coupled together in any desired configuration. Referring now to FIGS. 6A-D, a mobile biologics production system 400 includes a first mobile carrier 410a, a second mobile carrier 410b, and a third mobile carrier 410c (collectively referred to as “mobile carriers 410”). The mobile carriers 410 can be substantially similar to any one of the mobile carriers 110, 210, 310, or any other mobile carriers described herein. In some embodiments, each of the mobile carriers 410 can include a preparation room (e.g., the preparation room described with respect to mobile biologics system 200 or 300), a production sub-system (e.g., production sub-system 130, 230, or 330), and a purification sub-system (e.g., purification sub-system 160, 260, or 360). In some embodiments, the first mobile carrier 410a includes the preparation room, the second mobile carrier 410b includes the production sub-system, and the third mobile carrier 410c includes the purification sub-system, respectively. Each of the mobile carriers 410 can be trailered to the biologic production location and coupled together. The coupling can include coupling of electric, water and/or gas lines, as well as physical coupling using walkways 415 (e.g., enclosed walkways). The walkways 415 can, for example be disposed such that each walkway 415 surrounds a door included in the mobile carriers 410. In this manner, a user can move within the mobile carriers 410 without being exposed to the outside environment. The walkways 415 can be configured to be sealed from the outside environment and also provide an aseptic or axenic environment.

As shown in FIG. 6A, in a first configuration, the first mobile carrier 410a can be disposed parallel to the second mobile carrier 410b and coupled via the walkway 415. The third mobile carrier 410c can be disposed parallel to the second mobile carrier 410b and coupled to the second mobile carrier 410b by the walkway 415.

In a second configuration shown in FIG. 6B, the mobile carriers 410 can be parallel to each other but the second mobile carrier 410b can be offset from each of the first mobile carrier 410a and the third mobile carrier 410c. Each of the first mobile carrier 410a and the third mobile carrier 410c can be coupled to the second mobile carrier 410b by the walkway 415 as shown in FIG. 6B.

In a third configuration as shown in FIG. 6C, the first mobile carrier 410a and the second mobile carrier 410b can be disposed orthogonal to each other such that an end of the first mobile carrier 410a is coupled to a sidewall of the second mobile carrier 410b through the walkway 415. Similarly, a third mobile carrier 410c is disposed orthogonal to the second mobile carrier 410c and in line with the first mobile carrier 410a, such that an end of the third mobile carrier 410c is coupled to a sidewall of the second mobile carrier 410b. In a variation of the third configuration, the first mobile carrier 410a and the third mobile carrier 410c can be offset from each other.

In a fourth configuration shown in FIG. 6D, the first mobile carrier 410a can be disposed parallel to but offset from the second mobile carrier 410b and coupled by the walkway 415. The third mobile carrier 410c is disposed orthogonal to each of the first mobile carrier 410a and the second mobile carrier 410b and coupled to the second mobile carrier 410b using the walkway 415.

In some embodiments, in any of the first, second, third, and/or fourth configurations, each of the first mobile carrier 410a, the second mobile carrier 410b, and the third mobile carrier 410c can include the production sub-system, the purification sub-system, and optionally a preparation room, as described herein. In some embodiments, the first mobile carrier 410a can include a preparation room, the second mobile carrier can include production sub-system, and the third mobile carrier 410c includes a purification sub-system respectively, such that the purification sub-system is accessible only after passing through the production sub-system included in the second mobile carrier 410b. In some embodiments, the first mobile carrier 410a can include the production sub-system, the second mobile carrier 410b can include the preparation room, and the third mobile carrier 410c can include the purification sub-system. In such embodiments, a user can access either of the production sub-system or the purification sub-system through the preparation area. Thus, the user can avoid walking through the production sub-system to access the purification sub-system.

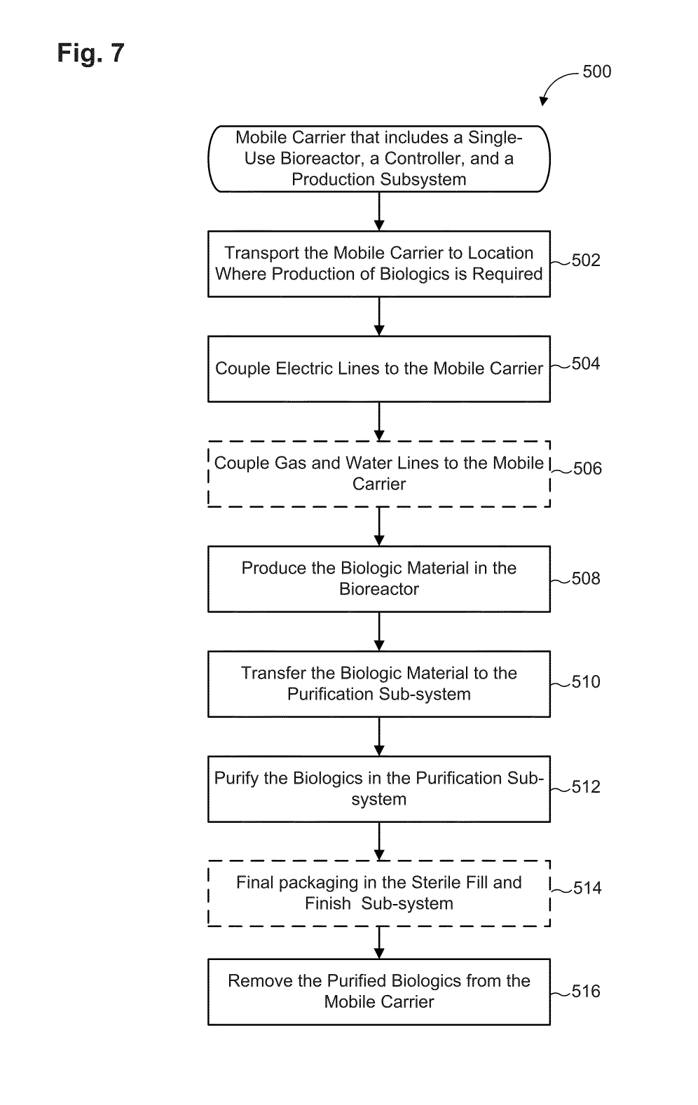

FIG. 7 illustrates a flow diagram showing an exemplary embodiment of a method 500 for producing biologics on demand. The method includes transporting a mobile carrier to a location where production of biologics is required 502. The mobile carrier can include any of the mobile carriers described herein, for example, the mobile carrier 110, 210, 310, or any other mobile carriers described herein. The mobile carrier includes a bioreactor (e.g., a single-use bioreactor or a multi-use bioreactor), for example, included in a production sub-system such as, for example, the production sub-system 130, 230, 330 or any other production sub-system described herein, and is configured to produce biologics, for example any biologics described herein. The mobile carrier also includes a controller, for example, the controller 150, 250, 350, or any other controller described herein, which is operable to control the operations of the bioreactors (e.g., bioreactors 232, 332 or any other bioreactor described herein) included in the production sub-system. The mobile carrier also includes a purification sub-system, for example, the purification sub-system 160, 260, 360 or any other purification sub-system described herein, configured to purify biologics to produce a finished biologics product. The mobile carrier optionally, also includes a sterile fill and finish sub-system, for example, the sterile fill and finish sub-system 170, 370, or any other purification sub-system described herein, configured to package biologics for use by the end user.

After the mobile carrier is transported to the desired location, electric lines are coupled to the mobile carrier 504. The electric lines can be used to supply electric power to the bioreactors, the purification sub-system, an HVAC sub-system (e.g., the HVAC sub-system 180, 280, or any other HVAC sub-system described herein), and/or a lighting sub-system (e.g., the lighting subs-system described with respect to the mobile biologics production system 100, 200, or 300) included in the mobile carrier. Optionally, gas and/or water lines can also be coupled to the mobile carrier 506 to supply gases and/or water required for growing the biologics in the bioreactors or for any other use thereof. The biologics are then produced in the bioreactor 508. The biologics can include, for example, veterinary proteins, human proteins, animal proteins, plant proteins, pharmaceutical proteins, renewable fuels, veterinary enzymes, human enzymes, animal enzymes, plant enzymes, pharmaceutical enzymes, microbial mass, microbial biomass, agricultural proteins, agricultural enzymes, biological control enzymes, biological control proteins, biological control microbes, virus particles, fatty acids, bioterrorism countermeasures, or any other biologics described herein. The produced biological material is then transferred to the purification sub-system 510. For example, the mobile carrier can include a pumping sub-system (e.g., the pumping sub-system described with reference to the mobile biologics production system 100, 200, or 300) for transferring the biologics produced in the bioreactors to the purification and/or sterile fill and finish sub-system. The biologics are purified in the purification sub-system 512, for example, by process columns (e.g., the process columns 362, or any other process columns described herein), and/or filtration devices (e.g., the filtration devices 364 or any other filtration devices described herein). The purified biologics are optionally transferred for final packaging in the sterile fill and finish subsystem 514, for example, sterile packaging in vats, vials, tubes, or any other containers. The purified biologics are then removed from the mobile carrier 516, for transportation to a user.

The following examples show mobile biologics production systems that can be trailered to a location where production of biologics is required. These examples are for illustrative purposes only and are not meant to limit the scope of the present disclosure.

### Example 1

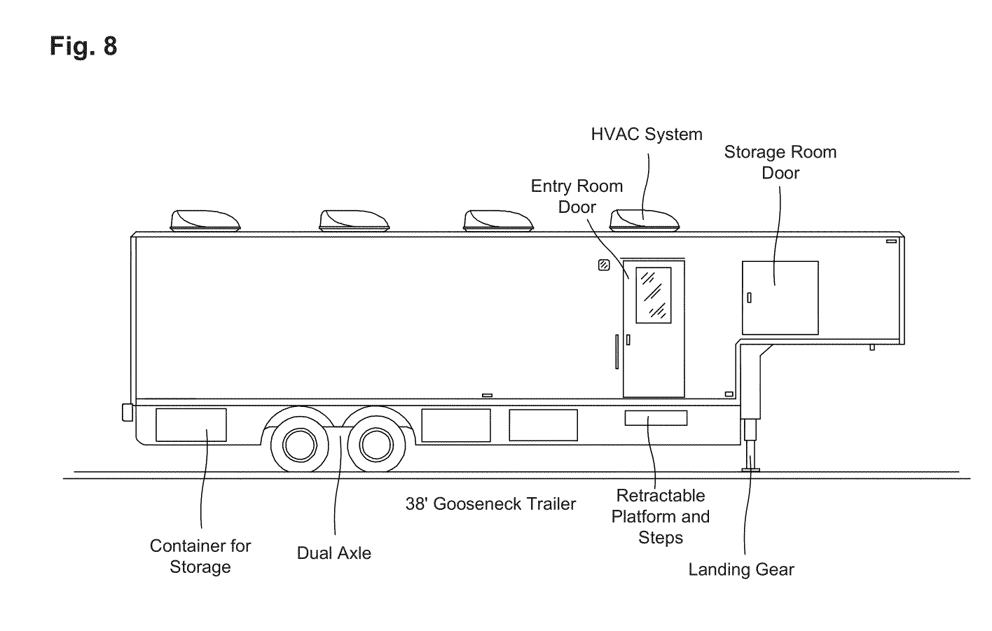

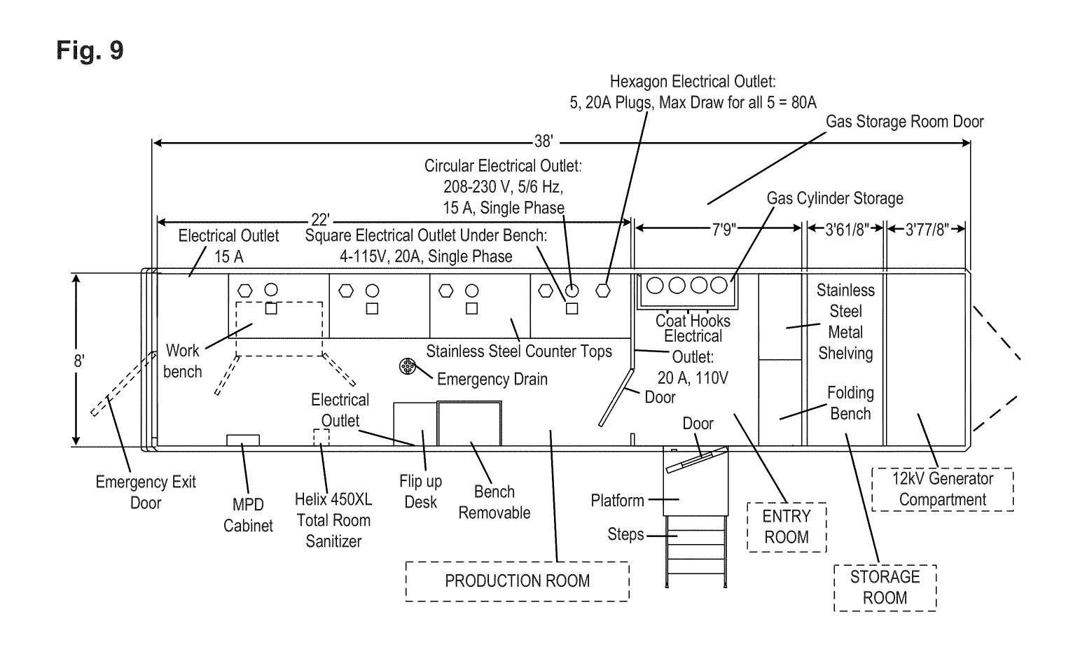

As shown in FIG. 8 and FIG. 9, a mobile biologics production system example 1 (also referred to as “the Ex. 1 system”) includes a mobile carrier which is a goose neck trailer. The mobile carrier has an overall length of about 38 feet, an overall width of about 8 feet and 6 inches, and an overall height of about 12 feet and 2 inches. The mobile carrier rides on dual axles and includes a retractable landing gear that can be engaged for deploying the mobile carrier at the location where biologics production is required. A plurality of containers are disposed in the undercarriage of the mobile carrier which can be used for storing equipment and/or supplies. An HVAC system is disposed on the roof of the mobile carrier.

The mobile carrier defines an internal volume that has a maximum height of about 8 feet. The internal volume is divided into a first compartment, a storage area, an entry room and a production room. The first compartment is located in a front portion of the mobile carrier and houses a 12 kV generator. The first compartment has a length of about 3 feet and 7⅞ inches. The storage area is also located in the front portion of the mobile carrier and includes a door. The storage area has a length of about 3 feet and 6⅛ inches, and can be used for storing equipment and/or supplies.

The entry room includes a door having a width of about 36 inches for accessing the entry room from an outside environment. The entry room has a length of about 7 feet and 9 inches. A retractable platform and steps are disposed at the base of the entry room door to enable a user to easily step into the entry room. The entry room also includes a folding bench which is about 48 inches wide and about 24 inches deep, and a stainless shelf which is also about 48 inches wide and 24 inches deep. The shelf can be used for storing PPEs, and/or other equipment and supplies. The entry room also includes a gas cylinder storage room for storing up to 4 gas cylinders and includes gas lines which run into the production room. The gas cylinder storage room includes a door to allow access from the outside environment. No access is provided to the gas cylinder storage room from the entry room for safety purposes. A plurality of coat hooks are disposed on a side wall of the entry room which can be used for hanging lab coats or personnel belongings. An electrical outlet, rated at about 20 A and 110 V is also provided on a side wall of the entry room. The entry room includes a second door which is about 36 inches wide and is configured to provide access to the production room in case of an emergency.

The production room has a length of about 22 feet and includes a plurality of stainless steel countertops, each having a width of about 36 inches and a length of about 56 inches. The countertops are provided with a back splash. A plurality of bioreactors (e.g., single-use or multi-use bioreactors) can be disposed on the countertops. A plurality of electrical outlets are disposed on the sidewall proximal to the countertops, including; (1) hexagonal electrical outlets which includes five 20 A plugs such that the max ampere draw for all 5 plugs is about 80 A, (2) single phase circle plugs rated at about 208-230 Vac, about ⅚ Hz, and about 15 A, and (3) single phase square plugs disposed beneath the countertops which are rated at about 4-115 V, and about 20 A. An about 4 inch wide, about 30 inch deep, and about 24 inch high work bench is removably disposed underneath the countertops such that the work bench can be pulled out from beneath the countertops as required by the user. The production room includes a bench seat having dimensions of about 36 inches by about 25 inches, and a flip-up desk having dimensions of about 24 inches by 24 inches, which are disposed along a sidewall of the mobile carrier. The production room includes an emergency drain on a floor of the production room. A helix 450 XL total room sanitizer are also disposed in the production room. The production room includes an emergency exit door to allow the user to access the outside environment.

### Example 2

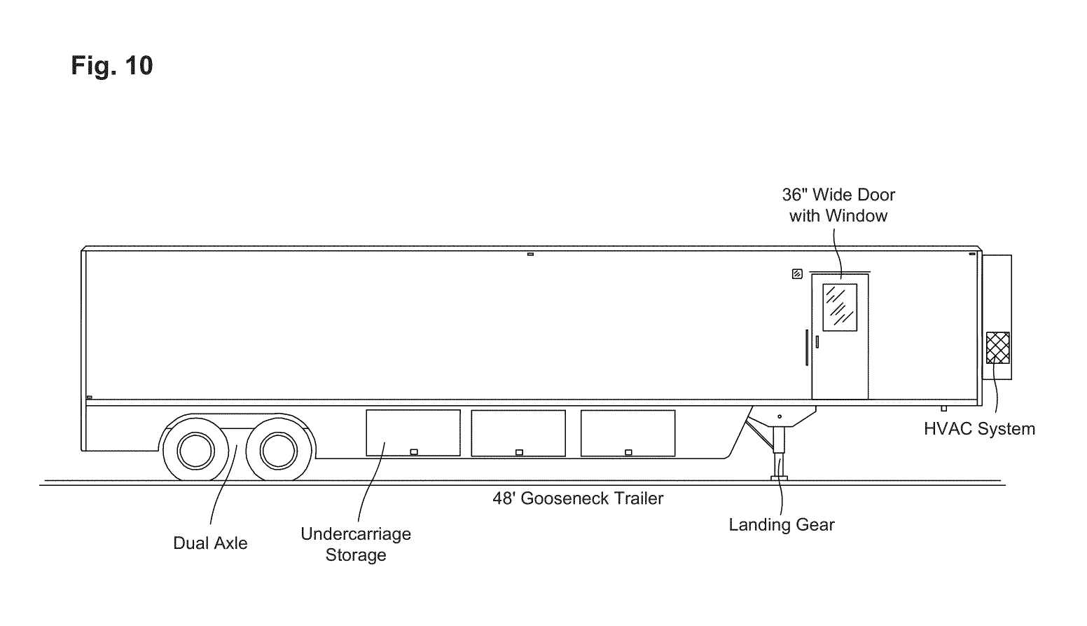

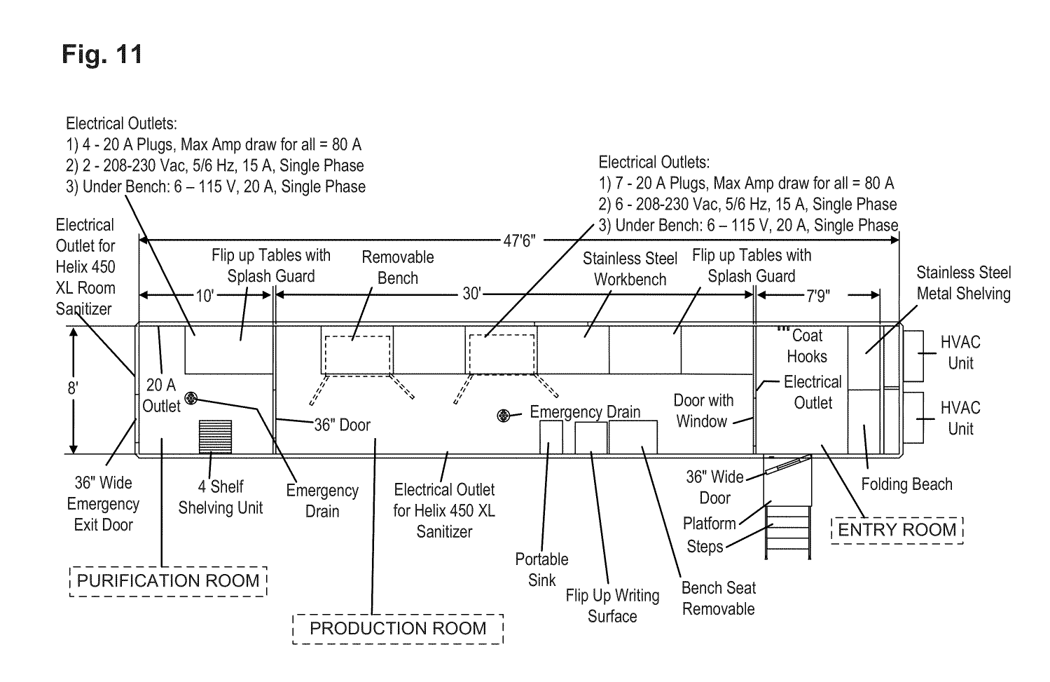

As shown in FIGS. 10 and 11, a mobile biologics production system example 2 (also referred to as “the Ex. 2 system”) includes a mobile carrier. The mobile carrier has an overall length of about 48 feet, an overall width of about 8 feet and 6 inches, and an overall height of about 12 feet and 10 inches. The mobile carrier rides on dual axles and includes a retractable landing gear that can be engaged for deploying the mobile carrier at the location where a production of biologics is required. A plurality of containers are disposed in the undercarriage of the mobile carrier which can be used for storing equipment and/or supplies. A plurality of gas containers are also disposed in the undercarriage containers.

The mobile carrier defines an interior volume that has a height of about 8 feet. The internal volume is divided into an entry room, a production room, and a purification room. An HVAC system is disposed in a front portion of the mobile carrier and is configured to provide hot and cold air into the internal volume of the mobile carrier as well as to purify the air by removing particles from the air.

The entry room includes a door which is about 36 inches wide for accessing the entry room from an outside environment. The entry room has a length of about 7 feet and 9 inches. A retractable platform and steps are disposed at the base of the entry room door to allow a user to easily step into the entry room. The entry room also includes a folding bench which is about 48 inches wide and about 24 inches deep, and stainless shelving which is also about 48 inches wide and about 24 inches deep which can be used for storing PPEs, and/or other equipment and supplies. A plurality of coat hooks are disposed on a side wall of the entry room which can be used for hanging lab coats or personnel belongings. A light switch, a light fixture, and an electrical outlet rated at about 20 A and about 110 V are also disposed on a side wall of the entry room. The entry room includes a second door which is about 36 inches wide. The second door defines a window and is configured to provide access to the production room. The sidewalls and ceiling of the entry room are coated with a smooth and washable paint. The floor of the entry room is coated with an epoxy resin.

The production room has a length of about 30 feet and includes stainless steel work benches and flip-up tables with splash guard such that the flip-up tables have a ground clearance of about 28 inches. A plurality of bioreactors (e.g., single-use or multi-use bioreactors) can be disposed on the stainless steel workbenches and/or the flip-up tables. A plurality of removable benches are disposed underneath the work benches. A plurality of electrical outlets are disposed on the sidewalls of the production room which include; (1) seven about 20 A plugs such that the max draw for all plugs is about 80 A, (2) six about 208-230 Vac, about ⅚ Hz, and about 15 A single phase plugs, (3) six about 115 V and about 20 A single phase plugs disposed under the work benches, and (4) an electrical outlet for a Helix 450 XL total room sanitizer that is also disposed in the production room. The production room also includes a removable bench seat having dimensions of about 36 inches by 25 inches, a flip-up writing surface and a portable sink disposed along a sidewall of the production room. An emergency drain is disposed on the floor of the production room which is closed when not in use. A plurality of gas lines run along a sidewall of the production room at a height of about 1 feet above the work benches. The production room also includes two or three light fixtures and a light switch which includes a rheostat disposed on a sidewall of the production room. The sidewalls and ceiling of the production room are coated with a smooth and washable paint while the floor is coated with an epoxy resin. The production room includes a door which is about 36 inches wide and connects the production room to the purification room.

The purification room is about 10 feet in length and includes a flip-up stainless steel table with back splash. The flip-up table is about 5 feet and 6 inches long, about 36 inches deep, and about 28 inches high. Purification equipment, for example a process column or a filtration device can be disposed on the flip-up table. A plurality of electrical outlets are disposed on the sidewalls of the purification room including; (1) four about 20 A plugs such that the max amp draw for all plugs is about 80 A, (2) two about 208-230 Vac, about ⅚ Hz, and about 15 A single phase plugs, (3) six about 115V, and about 20 A single phase plugs disposed on a portion of the sidewall underneath the workbenches, and (4) and an electrical outlet for the Helix 450 XL total room sanitizer. A four shelf, floor to ceiling shelving unit which is about 24 inches long and about 25 inches deep is also disposed in the purification room. An emergency drain is provided on the floor of the purification room which is closed when not in use. An emergency exit door which is about 36 inches wide is included in the purification room to allow a user to access the outside environment from the purification room in case of an emergency.

While various embodiments of the system, methods and devices have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and such modification are in accordance with the variations of the invention.

For example, in some embodiments, electric power to any of the mobile carriers described herein can be provided by an additional power generation mobile carrier that includes an industrial capacity power generator. The power generation mobile carrier can be transported to the biologics production location along with the biologics production mobile carrier and coupled to the biologics production mobile carrier on site.

In some embodiments, an additional portion can be included in any of the mobile biologics production system described herein, for example for packaging the product produced therein. In some embodiments, any of the mobile biologics production system that include a plurality of mobile carriers, for example the mobile biologics production system 400 or any other mobile biologics production system described herein, can include at least one mobile carrier dedicated for packaging the product.

In some embodiments, each of the interior sidewalls of any of the mobile carriers described herein can also be lined with panels made from a smooth and non-porous material, for example, stainless steel, vinyl, plastics, or any other suitable material, which can further be painted with a non-porous and easily washable material.

In some embodiments, any of the mobile carriers described herein can also include security cameras to monitor the interior volume, for example, the preparation room, the production sub-system, or the purification sub-system, or the outside environment of the mobile carrier.

Additionally, certain of the steps may be performed concurrently in a parallel process when possible, as well as performed sequentially as described above. The embodiments have been particularly shown and described, but it will be understood that various changes in form and details may be made.

1. A mobile biologics production system; comprising:
a mobile carrier configured to be trailered to a biologics production location, the mobile carrier defining an interior volume configured to provide an aseptic environment for manufacturing production and purification of biologics;
a production sub-system disposed in the interior volume, the production sub-system including a single-use bioreactor configured to grow at least one of microbes, mammalian cell lines, insect cell lines, plant cell lines, and algae;
a controller operable to control bioreactors having a working capacity less than about 100 liters;
a purification sub-system disposed in the interior volume and fluidically coupled to the production sub-system and configured to produce a finished biologic product; and
a sterile fill and finish sub-system is also disposed in the interior volume and fluidically coupled to the purification sub-system, the sterile fill and finish sub-system configured to produce a finished biologic product for dissemination to end-users.

2. The system of claim 1, wherein the single-use bioreactor is removably coupled to the production sub-system.

3. The system of claim 1, wherein the bioreactor is a multi-use bioreactor.

4. The system of claim 1, wherein the mobile carrier is a semi-trailer.

5. The system of claim 1, wherein the production sub-system and purification sub-system are configured to produce the finished biologic production to GLP standards.

6. The system of claim 1, wherein the production sub-system and purification sub-system are configured to produce the finished biologic production to GMP standards.

7. The system of claim 1, wherein the purification sub-system includes a process column.

8. The system of claim 1, wherein the purification sub-system includes a filtration device.

9. The system of claim 1, further comprising:
a lighting sub-system, the lighting sub-system including a rheostat configured to control light intensity.

10. The system of claim 1, further comprising:
an HVAC sub-system for heating and cooling the interior volume.

11. The system of claim 10, wherein the HVAC sub-system includes a filter to purify the air in the interior volume.

12. The system of claim 1, wherein the finish biologic product includes at least one of veterinary proteins, human proteins, animal proteins, plant proteins, pharmaceutical proteins, renewable fuels, veterinary enzymes, human enzymes, animal enzymes, plant enzymes, pharmaceutical enzymes, microbial mass, microbial biomass, agricultural proteins, agricultural enzymes, biological control enzymes, biological control proteins, biological control microbes, and virus particles.

13. A mobile biologics production system; comprising:
a first mobile carrier configured to be trailered to a biologics production location, the first mobile carrier defining an interior volume configured to provide an aseptic environment for manufacturing production and purification of biologics;
a second mobile carrier configured to be trailered to the biologics production location, the second mobile carrier defining an interior volume configured to provide an aseptic environment for manufacturing production and purification of biologics, the second mobile carrier configured to be coupleable to the first mobile carrier;
a production sub-system disposed in at least one of the interior volume of the first mobile carrier and the interior volume of the second mobile carrier, the production sub-system including a bioreactor configured to grow at least one of microbes, mammalian cell lines, insect cell lines, plant cell lines, and algae;
a controller operable to control bioreactors having a working capacity in the range of about 1 liter to about 100 liters;
a purification sub-system disposed in at least one of the interior volume of the first mobile carrier and the interior volume of the second mobile carrier and fluidically coupled to the production sub-system and configured to produce a finished biologic product; and
a sterile fill and finish sub-system is also disposed in the interior volume and fluidically coupled to the purification sub-system, the sterile fill and finish sub-system configured to produce a finished biologic product for dissemination to end-users.

14. The system of claim 13, wherein the bioreactor is at least one of a perfusion bioreactor, a wave bioreactor, a cylindrical bioreactor, a bag bioreactor, a moving bed bioreactor, a packed bag bioreactor, a fibrous bioreactor, a membrane bioreactor, a batch bioreactor, and a continuous bioreactor.

15. The system of claim 14, wherein the bioreactor is a single-use bioreactor.

16. The system of claim 15, wherein the bioreactor is a multi-use bioreactor.

17. The system of claim 13, wherein the bioreactor is coupled with at least one of a fermenter and a digester.

18. The system of claim 13, wherein the bioreactor has a capacity of at least about 1 L.

19. The system of claim 18, wherein the bioreactor has a capacity of at least about 10 L.

20. A method for producing biologics on demand, comprising:
transporting a mobile carrier to a location where production of biologics is required, the mobile carriers including a single-use bioreactor, a controller operable to the control the bioreactor, a purification sub-system, and a sterile fill and finish sub-system;
coupling electric lines to the mobile carrier;
producing biologic materials in the bioreactors;
transferring the biologic materials to the purification sub-system;
purifying the biologics in the purification sub-system; and
removing the purified biologics from the mobile carrier.
